SEQUENCE LISTING (6 sequences)

SEQ ID NO: 1 - protein, 361 aa, Streptomyces coelicolor
MHAFPHGTTATPTAIAVPPSLRLPVIEAAFPRQLHPYWPKLQETTRTWLLEKRLMPADKVEEYADGLCYTDLMAGYYLGAPDEVLQAIADYSAWFFVWDDRHDRDIVHGRAGAWRRLRGLLHTALDSPGDHLHHEDTLVAGFADSVRRLYAFLPATWNARFARHFHTVIEAYDREFHNRTRGIVPGVEEYLELRRLTFAHWIWTDLLEPSSGCELPDAVRKHPAYRRAALLSQEFAAWYNDLCSLPKEIAGDEVHNLGISLITHHSLTLEEAIGEVRRRVEECITEFLAVERDALRFADELADGTVRGKELSGAVRANVGNMRNWFSSVYWFHHESGRYMVDSWDDRSTPPYVNNEAAGEK

SEQ ID NO: 2 - protein, 337 aa, Streptomyces sp. UC5319
MPQDVDFHIPLPGRQSPDHARAEAEQLAWPRSLGLIRSDAAAERHLRGGYADLASRFYPHATGADLDLGVDLMSWFFLFDDLFDGPRGENPEDTKQLTDQVAAALDGPLPDTAPPIAHGFADIWRRTCEGMTPAWCARSARHWRNYFDGYVDEAESRFWNAPCDSAAQYLAMRRHTIGVQPTVDLAERAGRFEVPHRVFDSAVMSAMLQIAVDVNLLLNDIASLEKEEARGEQNNMVMILRREHGWSKSRSVSHMQNEVRARLEQYLLLESCLPKVGEIYQLDTAEREALERYRTDAVRTVIRGSYDWHRSSGRYDAEFALAAGAQGYLEELGSSAH

SEQ ID NO: 3 - protein, 546 aa, Matricaria chamomilla
MSLQENVIRPTANFPPSVWGDQFLTYDEREDQAGLEKVVEDLKDKVRQEILGTLDVPSQHTDLLRLIDSIQRLGIAYHFEEEIDRTLHHFYDAYGDNWTGGATSVWFRIMRQHGFFVSSDVFKSYKDKNGAFKEPLENDIVGFLELYEATYLRVPGEVILDDALVFTKGRLGEISNDPLWRNSIVSTQIIEALEQPVQKRLHRHEALRYITFYQQQASCNESLLKLAKLGFNLLQSLHKKELSQVYKWWKGFDVPTNLPYARNRMVECYFWSLSVFFEPKYSESRMFLAKVFAVETILDDTYDAFGTYEELEIFTAAVHRSSVTCLDALPKNYKLIYRIILSLYEDMEKILTKMGKAHHLNYIRNAMMEYIGCYLKEAKWANDEYTPTMEEHKEVTTVSSGYKFSLIASFAAMGDAITDETFKWALTMPPLAKACCVLCRVMDDIVTHKEEQERKHVASGIQCYMKEFDVTEQHVYDVFNAKVEDAWVEMNEESLKCKDVKRPVIMRVINLARAMDVLYKNKDHYTHVGPELINHIKSLVVDPIMA

SEQ ID NO: 4 - protein, 5 aa, Streptomyces coelicolor
  misc_feature (3)..(5): Xaa can be any naturally occurring amino acid, except for the Xaa at position 5 is Asp or Glu
DDXXX

SEQ ID NO: 5 - protein, 5 aa, Streptomyces coelicolor
  misc_feature (3)..(4): Xaa can be any naturally occurring amino acid
DDXXD

SEQ ID NO: 6 - protein, 5 aa, Streptomyces coelicolor
  misc_feature (3)..(4): Xaa can be any naturally occurring amino acid
DDXXE

(19) United States
(12) Patent Application Publication
(10) Pub. No.: US 2020/0239796 A1
(43) Pub. Date: Jul. 30, 2020

(54) HOST CELLS AND METHODS FOR PRODUCING TRICYCLIC SESQUITERPENES, AVIATION AND MISSILE FUEL PRECURSORS

(71) Applicant: The Regents of the University of California, Oakland, CA (US)

(72) Inventor: Taek Soon Lee, Berkeley, CA (US)

(73) Assignee: The Regents of the University of California, Oakland, CA (US)

(21) Appl. No.: 16/588,863

(22) Filed: Sep. 30, 2019

Related U.S. Application Data
(60) Provisional application No. 62/738,976, filed on Sep. 28, 2018.

(51) Int. Cl. C10L 1/04; C12P 15/00; C12P 5/00; C12N 1/20; C12N 1/16

(52) U.S. Cl. CPC C10L 1/04; C12P 15/00; C12P 5/005; C12N 1/20; C12R 1/19; C12Y 402/01136; C12Y 402/03007; C12Y 402/03037; C12N 1/16

(57) ABSTRACT

The present invention provides for a fuel compositions are provided comprising a hydrogenation product of a tricyclic sesquiterpene (epi-isozizaene, pentalenene, or α-isocomene) and a fuel additive. Methods of making and using the fuel compositions are also disclosed.

### CROSS-REFERENCE TO RELATED APPLICATIONS

This application claims priority to U.S. Provisional Patent Application Ser. No. 62/738,976, filed on Sep. 28, 2018, which is hereby incorporated by reference.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of production of tricyclic sesquiterpenes, which are aviation and missile fuel precursors.

### BACKGROUND OF THE INVENTION

Aviation fuels are an important target of biofuels research due to its high market demand and competitive price. Isoprenoids have been shown as good feedstocks for advanced renewable jet fuels with high energy density, heat of combustion, and cold-weather performance. Especially, sesquiterpene compounds C15 such as farnesene and bisabolene, have been identified as promising jet fuel candidates.

Several microbial platforms have been developed for advanced biofuel production [1-3]. The advanced biofuels, derived from higher alcohols, alkanes/alkenes, fatty acid esters and isoprenoids, are sustainable energy alternatives, with favorable properties and great market potential [3-5]. Among these biofuel candidates, C₁₀ and C₁₅ terpenes (monoterpenes and sesquiterpenes) have been increasingly used as jet fuel alternatives for commercial aviation and military purpose because of their structures, suitable carbon numbers and reactive olefin functionality, which collectively impart low freezing points and high energy densities [6-8]. For example, monoterpenes or monoterpenoids, such as pinenes [9], linalool [10] and limonene [11] have been used as biosynthetic precursors for aviation and missile fuels such as JP-10, RJ-4 and Jet A-1. Additionally, the hydrogenated sesquiterpene, farnesane, has been commercialized as a blend stock for jet fuel AMJ-700 [12]. Recently, there has been significant interest in multicyclic sesquiterpenes as next-generation jet fuel substitutes due to their high energy density and comparable cetane numbers [13]. For example, three sesquiterpenes, thujopsene, α-cedrene, and β-cedrene, were hydrogenated to generate a fuel blend with 12% higher volumetric net heat of combustion than conventional jet fuel [13].

### SUMMARY OF THE INVENTION

The present invention provides for a genetically modified host cell capable of producing one or more tricyclic sesquiterpenes, said genetically modified host cell comprising one or more tricyclic sesquiterpenes synthase. The tricyclic sesquiterpene is epi-isozizaene, pentalenene, or α-isocomene. In some embodiments, the genetically modified host cell comprises one or more enzymes of the mevalonate (MVA) pathway and/or the 2-C-methyl-D-erythritol 4-phosphate (MEP) pathway, and/or isopentenyl diphosphate (IPP) isomerase, and/or farnesyl diphosphate (FPP) synthase. In some embodiments, the one or more enzymes of the mevalonate (MVA) pathway, one or more enzymes of the 2-C-methyl-D-erythritol 4-phosphate (MEP) pathway, IPP isomerase, and/or FPP synthase are heterologous to the host cell. In some embodiments, the genetically modified host cell comprises one or more enzymes described in FIG. 1 herein.

The present invention provides for a mixture of tricyclic sesquiterpenes comprising epi-isozizaene, pentalenene, or α-isocomene, or a mixture thereof. In some embodiments, the mixture of tricyclic sesquiterpenes comprises epi-isozizaene and pentalenene. In some embodiments, the mixture of tricyclic sesquiterpenes comprises epi-isozizaene and α-isocomene. In some embodiments, the mixture of tricyclic sesquiterpenes comprises pentalenene and α-isocomene. In some embodiments, the mixture of tricyclic sesquiterpenes comprises epi-isozizaene, pentalenene, and α-isocomene.

The tricyclic sesquiterpene synthase is epi-isozizaene synthase (EIZS), pentalenene synthase (PentS), or α-isocomene synthase (MrTPS2). When the tricyclic sesquiterpene is epi-isozizaene, the tricyclic sesquiterpene synthase is epi-isozizaene synthase (EIZS). When the tricyclic sesquiterpene is pentalenene, the tricyclic sesquiterpene synthase is pentalenene synthase (PentS). When the tricyclic sesquiterpene is α-isocomene, the tricyclic sesquiterpene synthase is α-isocomene synthase (MrTPS2).

The present invention provides for a method for producing one or more tricyclic sesquiterpenes comprising: (a) providing a genetically modified host cell of the present invention, or a culture comprising the genetically modified host cell, (b) culturing the genetically modified host cell to produce one or more tricyclic sesquiterpenes, (c) optionally extracting or separating the produce one or more tricyclic sesquiterpenes from the culture, (d) hydrogenating the one or more tricyclic sesquiterpenes extracted or separated from the culture, and (e) optionally introducing a fuel additive to the extracted or separated one or more tricyclic sesquiterpenes. In some embodiments, the culture comprising the genetically modified host cell is a pure culture of the genetically modified host cell.

In some embodiments, the methods produce one or more tricyclic sesquiterpenes with a yield equal to or more than about 10, 20, 30, 40, 50, 60, or 70 mg/L of a tricyclic sesquiterpene. In some embodiments, the methods produce one or more tricyclic sesquiterpenes with a yield equal to or more than about 100, 200, 300, 400, 500, 600, or 700 mg/L of a tricyclic sesquiterpene. In some embodiments, the methods produce one or more tricyclic sesquiterpenes with a yield equal to or more than 77.5 mg/L of a tricyclic sesquiterpene, 727.9 mg/L epi-isozizaene, 780.3 mg/L pentalenene, and/or 77.5 mg/L α-isocomene.

The present invention provides for multicyclic sesquiterpenes, epi-isozizaene, pentalenene and α-isocomene, as novel jet fuel precursors. Multicyclic sesquiterpenes have higher energy density than previously known biojet fuel compounds. With a combined effort of expression heterologous mevalonate pathway and promoter engineering, 727.9 mg/L epi-isozizaene, 780.3 mg/L pentalenene and 77.5 mg/L α-isocomene are produced in Escherichia coli. The production of pentalenene is explored in more industrially favored Saccharomyces cerevisiae host and achieved 344 mg/L pentalenene as a proof of concept. More metabolic engineering efforts are currently ongoing, and this is the first report on the microbial production of tricyclic sesquiterpenes epi-isozizaene, pentalenene and α-isocomene with high production titer.

Bio-based platforms for advanced biofuels production have been developed using various synthetic biology and metabolic engineering technologies. The advanced biofuels, derived from higher alcohols, alkanes/alkenes, fatty acid esters and isoprenoids, are sustainable energy alternatives, with favorable properties and great market potential. Among those biofuel candidates, C10 and C15 isoprenoids (monoterpenes and sesquiterpenes) harboring compact structures, suitable carbon numbers and reactive olefin functionality, impart low freezing point and high energy density, thus have been increasingly used as jet fuel alternatives for commercial aviation and military purpose. For example, monoterpene pinenes (Harvey et al., 2010), linalool (Mendez-Perez et al., 2017) and limonene (Chuck and Donnelly, 2014) have been considered as biosynthetic precursors for aviation fuel JP-10, RJ-4 and Jet A-1 respectively. Additionally, the hydrogenated sesquiterpene, farnesane, has been commercialized as a blend stock for jet fuel AMJ-700 (Brennan et al., 2015). Recently, multicyclic sesquiterpenes are of interest in the development as next-generation jet fuel substitutes due to their high energy density and cetane numbers. Three sesquiterpenes thujopsene, α-cedrene, and β-cedrene, are hydrogenated to generate a fuel blend with 12% higher volumetric net heat of combustion than conventional jet fuel (Harrison and Harvey, 2017).

To expand the variety of multicyclic hydrocarbons as jet fuel alternatives, three novel tricyclic sesquiterpenes are produced: epi-isozizaene, pentalenene and α-isocomene. These sesquiterpenes are primarily identified as antibiotic metabolites. Epi-isozizaene and pentalenene are produced from several Streptomyces species (Aaron et al., 2010; Kim et al., 2013; Lin et al., 2006; Takamatsu et al., 2011), while α-isocomene is mainly isolated from plants such as Isocoma wright, Berkheya radulu and Matricaria recutita (Dauben and Walker, 1981; Irmisch et al., 2012; Paquette and Han, 1981). Their production and recovery efficiency from the natural producers, however, is very low when considering the quantity and the purity. In this invention, these multicyclic sesquiterpenes can be used as jet fuel blend and microbes can be engineered for scalable and more efficient production of these jet fuel candidates.

In previous researches, the synthases of epi-isozizaene (EIZS), pentalenene (PentS) and α-isocomene (MrTPS2) are identified (Cane et al., 1994; Quaderer et al., 2002; Rosano and Ceccarelli, 2014), and the in vitro activities of these terpene synthases to cyclize the farnesyl diphosphate (FPP) into cyclic sesquiterpenes are verified. However, little has been reported to synthesize these sesquiterpenes using conventional industrial hosts, such as S. cerevisiae and E. coli, to achieve decent production titer.

Three sesquiterpene synthases are expressed in E. coli separately to produce epi-isozizaene, pentalenene and α-isocomene. Both the endogenous methylerythritol phosphate (MEP) pathway and the heterologous mevalonate (MVA) pathway are engineered for efficient FPP supply in E. coli. Codon optimization of the sesquiterpene synthases and promoter engineering are performed to improve the terpene productions. This invention provides for a new class of jet and/or missile fuel compounds produced from a microbial system. This invention can be used to supply jet and/or missile fuel feedstocks from microbial system. Microbial production system is scalable and renewable alternative to the biofuel production.

Cyclic compounds are favored as jet and/or missile fuel due to high energy density, but it is not trivial to produce cyclic compound chemically. Nature has a good system to generate multicyclic compounds using enzymes that cyclize isoprenoid precursors to terpenes. Natural system for these products is mostly plants and some microbes, but they do not have advantage of scaling. Engineered microbial system has potential of scale-up and also allow improvement of the products profile by enzyme engineering.

The present invention also provides a fuel composition, the fuel composition comprising: (a) a tricyclic sesquiterpene and/or a hydrogenation product of a tricyclic sesquiterpene; and (b) a fuel additive. In one embodiment, the tricyclic sesquiterpene is epi-isozizaene, pentalenene, α-isocomene, or a mixture thereof. In some embodiments, the fuel composition further comprises α-zingiberene, β-sesquiphellandrene, α-bisabolene, β-bisabolene, γ-bisabolene, curcumene, gossonorol, or any monocyclic sesquiterpene taught in U.S. Pat. No. 9,109,175 (herein incorporated by reference), or a mixture thereof.

In one embodiment, the fuel additive that is mixed with the hydrogenation product of the tricyclic sesquiterpene is a chemical compound or component added to the fuel composition to alter the property of the fuel, e.g., to improve engine performance, fuel handling, fuel stability, or for contaminant control, etc. The nature and amount of the one or more additives depends on the desired use of the final fuel composition. Some nonlimiting examples of conventional fuel additives include antioxidants, thermal stability improvers, cetane improvers, stabilizers, cold flow improvers, combustion improvers, anti-foams, anti-haze additives, corrosion inhibitors, lubricity improvers, icing inhibitors, injector cleanliness additives, smoke suppressants, drag reducing additives, metal deactivators, dispersants, detergents, demulsifiers, dyes, markers, static dissipaters, biocides, and combinations thereof.

In some embodiments, the fuel composition of the present invention may further comprise a conventional fuel component derived from petroleum, coal, wood, or any other hydrocarbon source. Nonlimiting examples of conventional fuel components include, but are not limited to, diesel fuels, jet fuels, kerosene, gasoline, and Fischer-Tropsch derived fuels. In some embodiments, the conventional fuel component is derived from petroleum or coal. In certain embodiments, the fuel component is or comprises a diesel fuel, a jet fuel, kerosene, gasoline, or a combination thereof. In other embodiments, the fuel component is or comprises a distillate diesel fuel.

In certain embodiments, the fuel composition of the present invention is intended for use in diesel engines. In other embodiments, the fuel composition of the present invention is intended for use in jet engines and/or missile propulsion systems. As such, the fuel compositions disclosed herein can be used as a fuel for internal combustion engines such as gasoline engines, diesel engines, jet engines, and/or missile propulsion systems.

In another aspect, the present invention provides a genetically modified host cell that produces a tricyclic sesquiterpene via a mevalonate (MVA) pathway and/or 2-C-methyl-D-erythritol 4-phosphate (MEP) pathway, and IPP isomerase, and FPP synthase, wherein the genetically modified host cell comprises a heterologous nucleic acid comprising a nucleotide sequence encoding a tricyclic sesquiterpene synthase. In one embodiment, the host cell is a yeast, such as Saccharomyces cerevisiae. In another embodiment, the host cell is a bacteria, such as Escherichia species, such as Escherichia coli. In one embodiment, the nucleotide sequence encoding tricyclic sesquiterpene synthase gene is codon optimized for expression in Escherichia coli.

In yet another aspect, the present invention provides a vehicle comprising an internal combustion engine, a fuel tank connected to the internal combustion engine, and a fuel composition in the fuel tank, wherein the fuel composition is the fuel composition as disclosed herein (e.g., hydrogenated tricyclic sesquiterpene), wherein the fuel combustion is used to power the internal combustion engine. In one embodiment, the internal combustion engine is a diesel engine. In another embodiment, the internal combustion engine is a jet engine or missile propulsion system.

In a further aspect, the present invention provides a method of powering an engine comprising the step of combusting a fuel composition of the present invention in the engine. In one embodiment, the engine is a diesel engine. In another embodiment, the engine is a jet engine or a missile propulsion system.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. The heterologous mevalonate (MVA) pathway and the native 2-C-methyl-D-erythritol 4-phosphate (MEP) pathway for tricyclic sesquiterpene production. The heterologous MVA pathway from S. cerevisiae are overexpressed in E. coli to convert acetyl-CoA into farnesyl diphosphate (FPP). The endogenous MEP pathway consisting of 9 genes condensed pyruvate and G3P and contributes to FPP supplementation. FPP is converted into three sesquiterpenes by epi-isozizaene synthase (EIZS), pentalenene synthase (PentS) and α-isocomene synthase (MrTPS2). Pathway enzymes are AtoB: acetoacetyl-CoA thiolase, HMGS: HMG-CoA synthase, HMGR: HMG-CoA reductase, MK: mevalonate kinase, PMK: phosphomevalonate kinase, PMD: mevalonate diphosphate decarboxylase, DXS: 1-deoxy-D-xylulose 5-phosphate synthase, DXR: 1-deoxy-D-xylulose 5-phosphate reductoisomerase, ispD: 4-diphosphocytidyl-2C-methyl-D-erythritol synthase, ipk: 4-diphosphocytidyl-2-C-methyl-D-erythritol kinase, ispF: 2-C-methyl-D-erythritol 2,4-cyclodiphosphate synthase, ispG: 1-hydroxy-2-methyl-2-(E)-butenyl 4-diphosphate synthase, ispH: 1-hydroxy-2-methyl-butenyl 4-diphosphate reductase, idi: IPP isomerase, and ispA: FPP synthase. Pathway intermediates are MVA: mevalonate, G3P: glyceraldehyde 3-phosphate, MEP: 2-C-methyl-D-erythritol 4-phosphate, HMBPP: 1-hydroxy-2-methyl-2-(E)-butenyl 4-pyrophosphate, IPP: isopentenyl diphosphate, DMAPP: dimethylallyl diphosphate, and FPP: farnesyl diphosphate.

FIG. 2A. Gas chromatography-mass spectrometry (GC-MS) profile of biosynthetic sesquiterpenes. Retention time and fragmentation pattern of biosynthetic epi-isozizaene (RT: 7.32 min) [17].

FIG. 2B. Gas chromatography-mass spectrometry (GC-MS) profile of biosynthetic sesquiterpenes. Retention time and fragmentation pattern of produced pantalenene (RT: 6.56 min) [45].

FIG. 2C. Gas chromatography-mass spectrometry (GC-MS) profile of biosynthetic sesquiterpenes. Retention time and fragmentation pattern of α-isocomene (RT: 6.95 min)from MrTPS2 [20, 46].

FIG. 2D. Gas chromatography-mass spectrometry (GC-MS) profile of biosynthetic sesquiterpenes. Retention time and fragmentation pattern of a broad spectrum of products from MrTPS2. The mixed products include silphinene (RT: 6.67 min) [20, 46].

FIG. 2E. Gas chromatography-mass spectrometry (GC-MS) profile of biosynthetic sesquiterpenes. Retention time and fragmentation pattern of a broad spectrum of products from MrTPS2. The mixed products include modeph-2-ene (RT: 6.90 min) [20, 46].

FIG. 2F. Gas chromatography-mass spectrometry (GC-MS) profile of biosynthetic sesquiterpenes. Retention time and fragmentation pattern of a broad spectrum of products from MrTPS2. The mixed products include α-isocomene (RT: 6.95 min) [20, 46].

FIG. 2G. Gas chromatography-mass spectrometry (GC-MS) profile of biosynthetic sesquiterpenes. Retention time and fragmentation pattern of a broad spectrum of products from MrTPS2. The mixed products include β-isocomene (RT: 7.15 min) [20, 46].

FIG. 2H. Gas chromatography-mass spectrometry (GC-MS) profile of biosynthetic sesquiterpenes. Retention time and fragmentation pattern of a broad spectrum of products from MrTPS2. The mixed products include caryophyllene (RT: 7.25 min) [20, 46].

FIG. 2I. Gas chromatography-mass spectrometry (GC-MS) profile of biosynthetic sesquiterpenes. Retention time and fragmentation pattern of a broad spectrum of products from MrTPS2. The mixed products include α-humulene (RT: 7.47 min) [20, 46].

FIG. 3A. Sesquiterpene production with MEP pathway. (A) Epi-isozizaene production with strains, each harboring plasmid JBEI-15849, JBEI-15862 and JBEI-15866. Titers of compounds are measured by GC-MS at 48 and 72 hours, and data represent averages of three biological replicates.

FIG. 3B. Sesquiterpene production with MEP pathway. (B) Pentalenene production with strains, each harboring plasmid JBEI-15863, JBEI-15867 and JBEI-15858, respectively. Titers of compounds are measured by GC-MS at 48 and 72 hours, and data represent averages of three biological replicates.

FIG. 3C. Sesquiterpene production with MEP pathway. (C) α-isocomene production with strains, each harboring plasmid JBEI-15864, JBEI-15865 and JBEI-15859. Titers of compounds are measured by GC-MS at 48 and 72 hours, and data represent averages of three biological replicates.

FIG. 4A. Sesquiterpene production with the MVA pathway and promoter engineering. (A) Plasmid architecture. A two-plasmid system for sesquiterpene production. Plasmid 1 contains MVA pathway genes producing FPP from acetyl-CoA either under a P_{lacUV5} (inducible) or an FPP-responsive promoter P_{gadE} (dynamic) with a medium copy origin (p15A) and chloramphenicol resistance gene. Plasmid 2 contains a sesquiterpene synthase gene under either P_{trc}, P_{T7} or an FPP-responsive promoter P_{rstA} with a high copy origin (colE1) and ampicillin resistance gene.

FIG. 4B. Sesquiterpene production with the MVA pathway and promoter engineering. (B) Epi-isozizaene production. Titers of compounds are measured at 24, 48 and 72 hours. Promoters for both plasmid 1 and plasmid 2 are listed to describe strains as well as strain names in x-axis. Data represent averages of three biological replicates with standard deviation as error bars.

FIG. 4C. Sesquiterpene production with the MVA pathway and promoter engineering. (C) Pentalenene production. Titers of compounds are measured at 24, 48 and 72 hours. Promoters for both plasmid 1 and plasmid 2 are listed to describe strains as well as strain names in x-axis. Data represent averages of three biological replicates with standard deviation as error bars.

FIG. 4D. Sesquiterpene production with the MVA pathway and promoter engineering. (D) α-isocomene production. Titers of compounds are measured at 24, 48 and 72 hours. Promoters for both plasmid 1 and plasmid 2 are listed to describe strains as well as strain names in x-axis. Data represent averages of three biological replicates with standard deviation as error bars.

FIG. 5A. Pentalenene production in S. cerevisiae. (A) The S. cerevisiae host engineered for the production of antimalarial precursor amorphadiene is re-engineered to produce pentalenene by overexpressing pentalenene synthase (PentS) in a plasmid. ERG10: acetyl-CoA acetyltransferase, IDI1: isoprenyl diphosphate isomerase, ERG20: farnesyl disphosphate synthase. Genes in blue arrows (upc2-1, tHMGR, ERG20 and PentS) are overexpressed.

FIG. 5B. Pentalenene production in S. cerevisiae. (B) Production titer with CSM medium and YEP medium supplemented with 1.8% galactose/0.2% glucose.

FIG. 5C. Pentalenene production in S. cerevisiae. (C) Cell growth (OD₆₀₀) with CSM medium and YEP medium supplemented with 1.8% galactose/0.2% glucose.

FIG. 6. ¹H NMR spectrum of biosynthetic epi-isozizaene. ¹H NMR (600 MHz, CDCl₃): δ2.22 (ddp, J=17.0, 9.1, 1.2 Hz, 1H), 2.13-2.03 (m, 1H), 1.83 (dd, J=7.4, 5.3 Hz, 1H), 1.80-1.76 (m, 2H), 1.76-1.71 (m, 1H), 1.62-1.54 (m, 1H), 1.48 (dd, J=10.5, 5.3 Hz, 1H), 1.43 (t, J=1.5 Hz, 3H), 1.40 (d, J=10.0, 1.9 Hz, 1H), 1.38-1.34 (m, 1H), 1.26-1.21 (m, 1H), 1.18 (tdd, J=11.5, 3.4, 2.1 Hz, 1H), 1.00 (s, 3H), 0.98 (s, 3H), 0.92 (d, J=6.4 Hz, 3H).

FIG. 7. ¹³C NMR spectrum of biosynthetic epi-isozizaene. ¹³C NMR (151 MHz, CDCl₃): δ143.0, 127.5, 52.7, 47.2, 40.5, 39.7, 37.0, 32.5, 28.7, 28.4, 27.3, 25.1 24.4, 14.1, 12.9.

FIG. 8. ¹H NMR spectrum of biosynthetic pentalenene. ¹H NMR (600 MHz, CDCl₃): δ5.15 (h, J=1.6 Hz, 1H), 2.66 (ddh, J=9.3, 4.5, 2.1 Hz, 1H), 2.54 (d, J z, 1H), 1.86-1.80 (m, 1H), 1.78 (ddd, J=12.5, 6.1, 3.0 Hz, 1H), 1.73 (dd, J=13.1, 1.0 Hz, 1H), 1.62 (h, J=1.4 Hz, 4H), 1.37-1.23 (in, 4H), 1.18 (ddd, J=12.5, 5.1, 0.9 Hz, 1H), 0.98 (s, 3H), 0.98 (s, 3H), 0.90 (d, J=7.1 Hz, 3H).

FIG. 9. ¹³C NMR spectrum of biosynthetic pentalenene. ¹³C NMR (151 MHz, CDCl₃): δ140.5, 129.5, 64.7, 62.0, 59.3.48.9, 46.8, 44.5, 40.5, 33.5, 29.9, 29.1, 27.5, 17.0, 15.5.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host microorganism; or (b) the structure or molecule may be naturally found in a given host microorganism, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host microorganism, wherein the expression vector contains a nucleic acid sequence coding for an enzyme that is not normally found in a host microorganism. With reference to the host microorganism's genome, then, the nucleic acid sequence that codes for the enzyme is heterologous.

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host microorganism or cell. Only when the sequence of nucleic acids becomes stably replicated by the cell does the host microorganism or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiments, the method comprises culturing the genetically modified host cell with exogenously provided MVA or MEP pathway intermediate, or a suitable carbon source. When the method comprises culturing the genetically modified host cell with a suitable carbon source, the genetically modified host cell is capable of synthesizing FPP using native enzymes and native biosynthetic pathway and/or a heterologous biosynthetic pathway residing on one or more nucleic acids in the host cell, wherein the one or more nucleic acids are on one or more vectors or stably integrated into a host cell chromosome. Suitable carbon sources which the host cell is capable of uptaking and metabolizing. Such carbon sources include but are not limited to sugars, such as monosaccharides, such as glucose.

In some embodiments, the method comprises: (a) introducing a nucleic acid construct encoding an enzyme capable of catalyzing the production of a tricyclic sesquiterpene into a genetically modified host cell; and (b) culturing the genetically modified host cell under a suitable condition such that the enzyme is expressed in the host cell; such that the culturing results in the genetically modified host cell producing the desired products.

In some embodiments, the host cell is capable of endogenously producing acetyl-CoA, pyruvate, G3P, any of the intermediate products of the MVA and/or MEP pathways, IPP, DMAPP, and/or FPP, either by native enzymes, or a heterologous enzymes, which genes are introduced into the host cell.

In some embodiments of invention, the method further comprises the step of extracting, separating, or recovering the produced tricyclic sesquiterpene, wherein the recovering step is concurrent or subsequent to the culturing step.

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of tricyclic sesquiterpene ensured. When added, the intermediate is present in an excess amount in the culture medium.

Any means for recovering the tricyclic sesquiterpene from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC). Once the tricyclic sesquiterpene is recovered, modification, such as hydrogenation, may be carried out on the tricyclic sesquiterpene.

In some embodiments, the epi-isozizaene synthase (EIZS), or a homologous enzyme thereof, has an amino acid sequence having at least 70% identity to the amino acid sequence of Streptomyces coelicolor epi-isozizaene synthase which is as follows:

```
(SEQ ID NO: 1)
                10 20 30 40
MHAFPHGTTA TPTAIAVPPS LRLPVIEAAF PRQLHPYWPK
                50 60 70 80
LQETTRTWLL EKRLMPADKV EEYADGLCYT DLMAGYYLGA
                90 100 110 120
PDEVLQAIAD YSAWFFVWDD RHDRDIVHGR AGAWRRLRGL
              130 140 150 160
LHTALDSPGD HLHHEDTLVA GFADSVRRLY AFLPATWNAR
              170 180 190 200
FARHFHTVIE AYDREFHNRT RGIVPGVEEY LELRRLTFAH
              210 220 230 240
WIWTDLLEPS SGCELPDAVR KHPAYRRAAL LSQEFAAWYN
              250 260 270 280
DLCSLPKEIA GDEVHNLGIS LITHHSLTLE EAIGEVRRRV
              290 300 310 320
EECITEFLAV ERDALRFADE LADGTVRGKE LSGAVRANVG
              330 340 350 360
NMRNWFSSVY WFHHESGRYM VDSWDDRSTP PYVNNEAAGE K
```

In some embodiments, the epi-isozizaene synthase comprises the amino acid sequence Asp-Asp-Xaa-Xaa-Asp/Glu (DDXXD/E) motif (SEQ ID NO:4) is important for the catalytic activity of epi-isozizaene synthase, presumably through binding to Mg²⁺.

In some embodiments, the pentalenene synthase (PentS), or a homologous enzyme thereof, has an amino acid sequence having at least 70% identity to the amino acid sequence of Streptomyces sp. UC5319 pentalenene synthase which is as follows:

(SEQ ID NO: 2)
MPQDVDFHIPLPGRQSPDHARAEAEQLAWPRSLGLIRSDAAAER
HLRGGYADLASRFYPHATGADLDLGVDLMSWFFLFDDLFDGPRGENPED
TKQLTDQVAAALDGPLPDTAPPIAHGFADIWRRTCEGMTPAWCARSARH
WRNYFDGYVDEAESRFWNAPCDSAAQYLAMRRHTIGVQPTVDLAERAGR
FEVPHRVFDSAVMSAMLQIAVDVNLLLNDIASLEKEEARGEQNNMVMIL
RREHGWSKSRSVSHMQNEVRARLEQYLLLESCLPKVGEIYQLDTAEREA
LERYRTDAVRTVIRGSYDWHRSSGRYDAEFALAAGAQGYLEELGSSAH

In some embodiments, the pentalenene synthase comprises the amino acid sequence Asp-Asp-Xaa-Xaa-Asp/Glu (DDXXD/E) motif (SEQ ID NO:4) is important for the catalytic activity of pentalenene synthase, presumably through binding to Mg²⁺.

In some embodiments, the α-isocomene synthase (MrTPS2), or a homologous enzyme thereof, has an amino acid sequence having at least 70% identity to the amino acid sequence of Matricaria chamomilla α-isocomene synthase which is as follows:

```
(SEQ ID NO: 3)
                10 20 30 40
MSLQENVIRP TANFPPSVWG DQFLTYDERE DQAGLEKVVE
                50 60 70 80
DLKDKVRQEI LGTLDVPSQH TDLLRLIDSI QRLGIAYHFE
                90 100 110 120
EEIDRTLHHF YDAYGDNWTG GATSVWFRIM RQHGFFVSSD
              130 140 150 160
VFKSYKDKNG AFKEPLENDI VGFLELYEAT YLRVPGEVIL
              170 180 190 200
DDALVFTKGR LGEISNDPLW RNSIVSTQII EALEQPVQKR
              210 220 230 240
LHRHEALRYI TFYQQQASCN ESLLKLAKLG FNLLQSLHKK
              250 260 270 280
ELSQVYKWWK GFDVPTNLPY ARNRMVECYF WSLSVFFEPK
              290 300 310 320
YSESRMFLAK VFAVETILDD TYDAFGTYEE LEIFTAAVHR
              330 340 350 360
SSVTCLDALP KNYKLIYRII LSLYEDMEKI LTKMGKAHHL
              370 380 390 400
NYIRNAMMEY IGCYLKEAKW ANDEYTPTME EHKEVTTVSS
              410 420 430 440
GYKFSLIASF AAMGDAITDE TFKWALTMPP LAKACCVLCR
              450 460 470 480
VMDDIVTHKE EQERKHVASG IQCYMKEFDV TEQHVYDVFN
              490 500 510 520
AKVEDAWVEM NEESLKCKDV KRPVIMRVIN LARAMDVLYK
              530 540
NKDHYTHVGP ELINHIKSLV VDPIMA
```

In some embodiments, the α-isocomene synthase comprises the amino acid sequence Asp-Asp-Xaa-Xaa-Asp/Glu (DDXXD/E) motif (SEQ ID NO:4) is important for the catalytic activity of α-isocomene synthase, presumably through binding to Mg²⁺.

In some embodiments, the Asp-Asp-Xaa-Xaa-Asp/Glu (DDXXD/E) motif (SEQ ID NO:4) is Asp-Asp-Xaa-Xaa-Asp (DDXXD) (SEQ ID NO:5) or Asp-Asp-Xaa-Xaa-Glu (DDXXE) (SEQ ID NO:6).

### Host Cells

The host cells of the present invention are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

Any prokaryotic or eukaryotic host cell may be used in the present method so long as it remains viable after being transformed with a sequence of nucleic acids. Generally, although not necessarily, the host cell is a yeast or a bacterium. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of bacterial host cells include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. In some embodiments, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway. Suitable eukaryotic cells include, but are not limited to, fungal, insect or mammalian cells. Suitable fungal cells are yeast cells, such as yeast cells of the Saccharomyces genus.

References cited:

1. Connor M R, Liao J C. Microbial production of advanced transportation fuels in non-natural hosts. Curr Opin Biotechnol 2009;20:307-315.
2. Tian T, Lee T S. Advanced Biodiesel and Biojet Fuels from Lignocellulosic Biomass. 2017:1-25.
3. Zhang F, Rodriguez S, Keasling J D. Metabolic engineering of microbial pathways for advanced biofuels production. Curr Opin Biotechnol. 2011;22:775-783.
4. Lee S K, Chou H, Ham T S, Lee T S, Keasling J D. Metabolic engineering of microorganisms for biofuels production: from bugs to synthetic biology to fuels. Curr Opin Biotechnol. 2008;19:556-563.
5. Rottig A, Wenning L, Broker D, Steinbuchel A. Fatty acid alkyl esters: perspectives for production of alternative biofuels. Appl Microbiol Biotechnol. 2010;85:1713-1733.
6. Beller H R, Lee T S, Katz L. Natural products as biofuels and bio-based chemicals: fatty acids and isoprenoids. Nat Prod Rep. 2015;32:1508-1526.
7. George K W, Alonso-Gutierrez J, Keasling J D, Lee T S. Isoprenoid drugs, biofuels, and chemicals—artemisinin, farnesene, and beyond. Adv Biochem Eng Biotechnol. 2015;148:355-389.
8. Phulara S C, Chaturvedi P, Gupta P. Isoprenoid-Based Biofuels: Homologous Expression and Heterologous Expression in Prokaryotes. Appl Environ Microbiol. 2016;82:5730-5740.
9. Harvey B G, Wright M E, Quintana R L: High-Density Renewable Fuels Based on the Selective Dimerization of Pinenes. Energ & Fuels. 2010;24:267-273.
10. Mendez-Perez D, Alonso-Gutierrez J, Hu Q, Molinas M, Baidoo E E K, Wang G, et al. Production of jet fuel precursor monoterpenoids from engineered Escherichia coli. Biotechnol Bioeng. 2017;114:1703-1712.
11. Chuck C J, Donnelly J. The compatibility of potential bioderived fuels with Jet A-1 aviation kerosene. Appl Energ. 2014;118:83-91.
12. Brennan T C, Williams T C, Schulz B L, Palfreyman R W, Kromer J O, Nielsen L K. Evolutionary Engineering Improves Tolerance for Replacement Jet Fuels in Saccharomyces cerevisiae. Appl Environ Microbiol. 2015;81:3316-3325.
13. Harrison K W, Harvey B G. Renewable high density fuels containing tricyclic sesquiterpanes and alkyl diamondoids. Sust Energ Fuels. 2017;1:467-473.
14. Zhao B, Lin X, Lei L, Lamb D C, Kelly S L, Waterman M R, et al. Biosynthesis of the sesquiterpene antibiotic albaflavenone in Streptomyces coelicolor A3(2). J Biol Chem 2008, 283:8183-8189.
15. Aaron J A, Lin X, Cane D E, Christianson D W. Structure of epi-isozizaene synthase from Streptomyces coelicolor A3(2), a platform for new terpenoid cyclization templates. Biochem. 2010;49:1787-1797.
16. Kim Y-J, Yoon Y, Lee H-Y. An asymmetric total synthesis of (+)-pentalenene. Tetrahedron. 2013;69:7810-7816.
17. Lin X, Hopson R, Cane D E: Genome Mining in Streptomyces coelicolor: Molecular Cloning and Characterization of a New Sesquiterpene Synthase. J Am Chem Soc. 2006;128:6022-6023.
18. Takamatsu S, Lin X, Nara A, Komatsu M, Cane D E, Ikeda H. Characterization of a silent sesquiterpenoid biosynthetic pathway in Streptomyces avermitilis controlling epi-isozizaene albaflavenone biosynthesis and isolation of a new oxidized epi-isozizaene metabolite. Microb Biotechnol. 2011;4:184-191.
19. Dauben M G, Walker D M. Formal Total Synthesis of (±)-isocomene. J Org Chem. 1981;46:1103-1108.
20. Irmisch S, Krause S, T, Kunert G, Gershenzon J, Degenhardt J, Köllner T, G.. The organ-specific expression of terpene synthase genes contributes to the terpene hydrocarbon composition of chamomile essential oils. BMC Plant Biology. 2012;12:1471-2229.
21. Paquette L A, Han Y K. Total Synthesis of (±)-isocomene, a Naturally Occurring Triquinane. J Am Chem Soc. 1981;103:1835-1838.
22. Cane D E, Sohng J K, Lamberson C R, Rudnicki S M, Wu Z, Lloyd M D, et al. Pentalenene Synthase. Purification, Molecular Cloning, Sequencing, and High-Level Expression in Escherichia coli of a Terpenoid Cyclase from Streptomyces UC53191. Biochem. 1994;33:5846-5857.
23. Quaderer R, Omura S, Ikeda H, Cane D E. Pentalenene Synthase. Analysis of Active Site Residues by Site-Directed Mutagenesis. J Am Chem Soc. 2002;124:7681-7689.
24. Rosano G L, Ceccarelli E A. Recombinant protein expression in Escherichia coli: advances and challenges. Front Microbiol. 2014;5:172.
25. Dahl R H, Zhang F, Alonso-Gutierrez J, Baidoo E, Batth T S, Redding-Johanson A M. Engineering dynamic pathway regulation using stress-response promoters. Nat Biotechnol. 2013;31:1039-1046.

### 26. M. J. Frisch G W T, H. B. Schlegel, G. E. Scuseria, M. A. Robb, J. R. Cheeseman, G. Scalmani, et al.. Gaussian 09, Revision A.1. Gaussian Inc.: Wallingford, Conn.; 2009.

27. Repetto S L, Patel R, Johnson T, Costello J F, Lam J K W, Chuck C J. Dual Action Additives for Jet A-1: Fuel Dehydrating Icing Inhibitors. Energ & Fuels. 2016;30:9080-9088.
28. Li R, Chou W K, Himmelberger J A, Litwin K M, Harris G G, Cane D E et al. Reprogramming the chemodiversity of terpenoid cyclization by remolding the active site contour of epi-isozizaene synthase. Biochem. 2014;53:1155-1168.
29. Martin J V, Yoshikuni Y, Keasling D J. The In Vivo Synthesis of Plant sesquiterpene by Escherichia coli. Biotechnol. and bioeng. 2001;75:7.
30. Martin J V, Pitera J D, Withers T S, Newman D J D, Keasling D J. Engineering a mevalonate pathway in Escherichia coli for production of terpenoids. Nat. Biotechnol. 2003;21:7.
31. Peralta-Yahya P P, Ouellet M, Chan R, Mukhopadhyay A, Keasling J D, Lee T S. Identification and microbial production of a terpene-based advanced biofuel. Nat Commun. 2011;2:1-8.
32. Ro D K, Paradise E M, Ouellet M, Fisher K J, Newman K L, Ndungu J M, et al. Production of the antimalarial drug precursor artemisinic acid in engineered yeast. Nat. 2006;440:940-943.
33. Kirby J, Keasling J D. Metabolic engineering of microorganisms for isoprenoid production. Nat Prod Rep. 2008;25:656-661.
34. Harvey B G, Meylemans H A, Gough R V, Quintana R L, Garrison M D, Bruno T J. High-density biosynthetic fuels: the intersection of heterogeneous catalysis and metabolic engineering. Phys Chem Chem Phys. 2014;16:9448-9457.
35. Wang C, Yoon S H, Jang H J, Chung Y R, Kim J Y, Choi E S, et al. Metabolic engineering of Escherichia coli for alpha-farnesene production. Metab Eng. 2011;13:648-655.
36. Yang X, Nambou K, Wei L, Hua Q. Heterologous production of alpha-farnesene in metabolically engineered strains of Yarrowia lipolytica. Bioresour Technol. 2016;216:1040-1048.
37. Zhu F, Zhong X, Hu M, Lu L, Deng Z, Liu T. In vitro reconstitution of mevalonate pathway and targeted engineering of farnesene overproduction in Escherichia coli. Biotechnol Bioeng. 2014;111:1396-1405.
38. Klein-Marcuschamer D, Turner C, Allen M, Gray P, Dietzgen R G, Gresshoff P M, et al. Technoeconomic analysis of renewable aviation fuel from microalgae, Pongamia pinnata, and sugarcane. Biofuels, Biopro and Bioref. 2013;7:416-428.
39. Gibson D G, Young L, Chuang R Y, Venter J C, Hutchison C A, 3rd, Smith H O. Enzymatic assembly of DNA molecules up to several hundred kilobases. Nat Methods. 2009;6:343-345.
40. Morris U. Separations of lipids by silver ion chromatography. J of lipid research. 1966;7:717-732.
41. Almatarneh M H, Al-Shamaileh E, Ahmad Z M, Abu-Saleh A A A A, Elayan I A. A Computational Study of the Ozonolysis of Phenanthrene. Acta Physica Polonica A. 2017;132:1149-1156.
42. da Silva G, Bozzelli J W, Sebbar N, Bockhorn H. Thermodynamic and ab initio analysis of the controversial enthalpy of formation of formaldehyde. Chemphyschem. 2006;7:1119-1126.
43. Qu R, Liu H, Feng M, Yang X, Wang Z. Investigation on Intramolecular Hydrogen Bond and Some Thermodynamic Properties of Polyhydroxylated Anthraquinones. J. of Chemical & Eng Data 2012, 57:2442-2455.
44. Redding-Johanson A M, Batth T S, Chan R, Krupa R, Szmidt H L, Adams P D, et al. Targeted proteomics for metabolic pathway optimization: application to terpene production. Metab Eng. 2011;13:194-203.
45. Agger S, Lopez-Gallego F, Schmidt-Dannert C. Diversity of sesquiterpene synthases in the basidiomycete Coprinus cinereus. Mol Microbiol. 2009;72:1181-1195.
46. Hong Y J, Irmisch S, Wang S C, Garms S, Gershenzon J, Zu L, et al. Theoretical and Experimental Analysis of the Reaction Mechanism of MrTPS2, a Triquinane-Forming Sesquiterpene Synthase from Chamomile. Chem Euro J. 2013;19:11.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Renewable Production of High Density Jet Fuel Precursor Sesquiterpenes from Escherichia Coli

Aviation fuels are an important target of biofuels research due to their high market demand and competitive price. Isoprenoids have been demonstrated as good feedstocks for advanced renewable jet fuels with high energy density, high heat of combustion, and excellent cold-weather performance. In particular, sesquiterpene compounds (C₁₅) such as farnesene and bisabolene, have been identified as promising jet fuel candidates. Three sesquiterpenes—epi-isozizaene, pentalenene and α-isocomene—are explored as novel jet fuel precursors. Through heterologous MVA pathway expression and promoter engineering, 727.9 mg/L epi-isozizaene, 780.3 mg/L pentalenene and 77.5 mg/L α-isocomene are produced in Escherichia coli and 344 mg/L pentalenene in Saccharomyces cerevisiae. A dynamic autoinduction system is introduced using previously identified FPP-responsive promoters for inducer-free production and comparable amounts of each compound are achieved. Tricyclic sesquiterpenes epi-isozizaene, pentalenene and α-isocomene, promising jet fuel feedstocks at high production titers, providing novel, sustainable alternatives to petroleum-based jet fuels are produced.

Three novel tricyclic sesquiterpenes: epi-isozizaene, pentalenene and α-isocomene are suitable multicyclic hydrocarbons as jet fuel alternatives. These sesquiterpenes were primarily identified as antibiotic metabolites [14]. Epi-isozizaene and pentalenene are produced by several Streptomyces species [15-18], while α-isocomene is mainly isolated from plants such as Isocoma wright, Berkheya radulu and Matricaria recutita [19-21]. Their production and recovery efficiency from the natural producers, however, is very low when considering the quantity and the purity. These multicyclic sesquiterpenes can be used as jet fuel blending agents. Microbes are engineered for the scalable production of these jet fuel candidates.

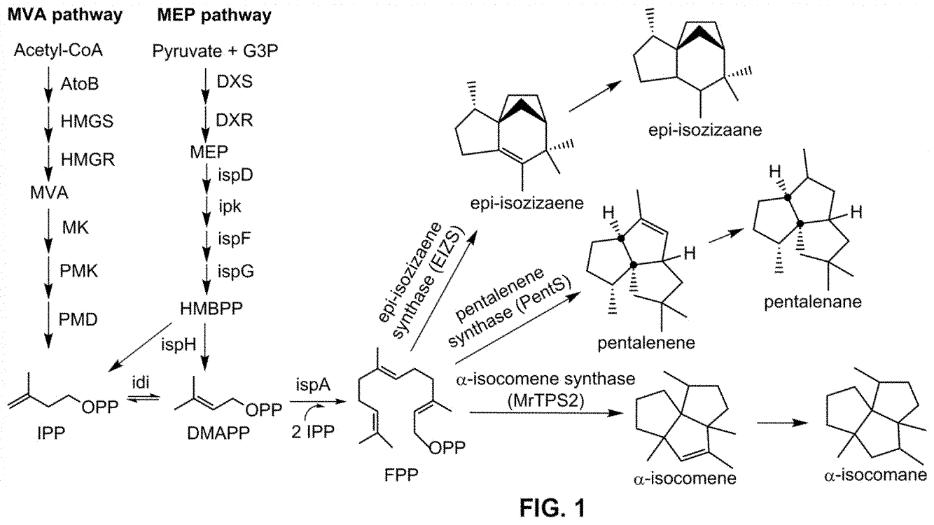

In previous research, the epi-isozizaene (EIZS), pentalenene (PentS) and α-isocomene (MrTPS2) synthases were identified [22-24], and their in vitro activities on farnesyl diphosphate (FPP) were verified. Despite these findings, little has been reported on the synthesis of these sesquiterpenes using conventional industrial hosts, such as S. cerevisiae and E. coli, to achieve reasonable product titers. These three sesquiterpene synthases are expressed in E. coli separately to produce epi-isozizaene, pentalenene and α-isocomene. Both the endogenous methylerythritol phosphate (MEP) pathway and the heterologous mevalonate (MVA) pathway are utilized for efficient FPP supply in E. coli (FIG. 1). Each of the sesquiterpene synthases are codon-optimized and their promoters are engineered to boost sesquiterpene production. To reduce production costs associated with chemical inducers and to minimize human supervision during fermentation process, an inducer-free system for production of these sesquiterpenes by introducing previously identified FPP-responsive dynamic promoters is developed [25]. Finally, the pathway for pentalenene production is introduced into S. cerevisiae, an important industrial host, and demonstrated production levels comparable to those achieved using E. coli.

### Results

Verification of Tricyclic Sesquiterpene Production in E. coli

For microbial production of the three jet fuel precursor candidates, the sesquiterpene synthases responsible for production of these sesquiterpenes were identified in the literature. Epi-isozizaene synthase (EIZS) from Streptomyces coelicolor [17, 28], pentalenene synthase (PentS) from Streptomyces UC5319 [22, 23], and α-isocomene synthase (MrTPS2) from Matricaria recutita (chamomile) [20], are selected among those reported terpene synthases, and expressed in E. coli after the gene sequences are codon-optimized to generate coEIZS, coPentS and coMrTPS2. Solubility of these codon-optimized enzymes is compared with the original EIZS without codon-optimization as a control. All terpene synthases tested show clear bands on SDS-PAGE gel in the whole cell lysate and supernatant, suggesting that they are soluble and well expressed in E. coli.

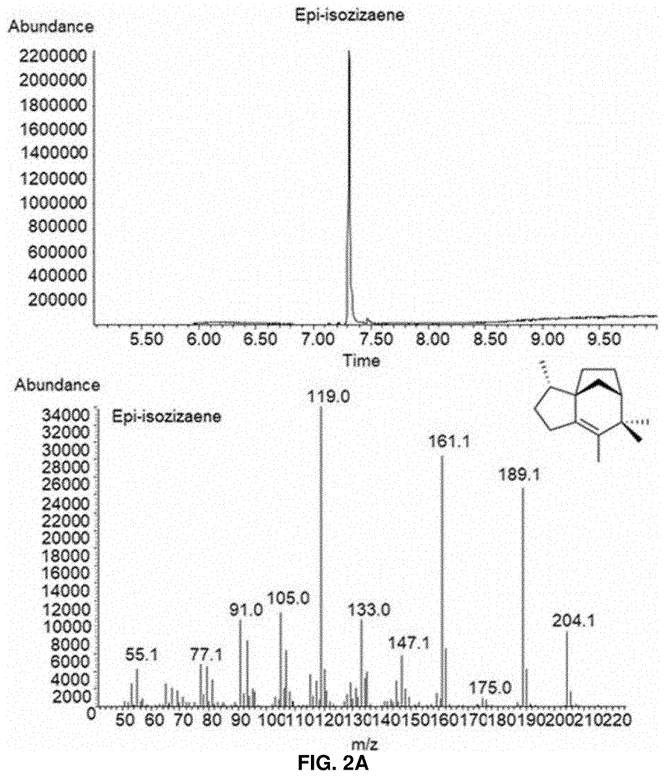

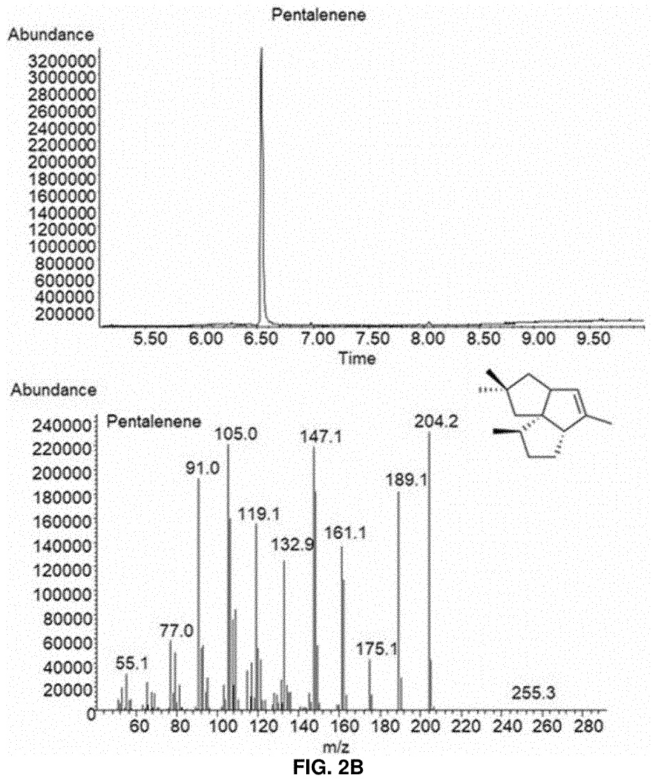

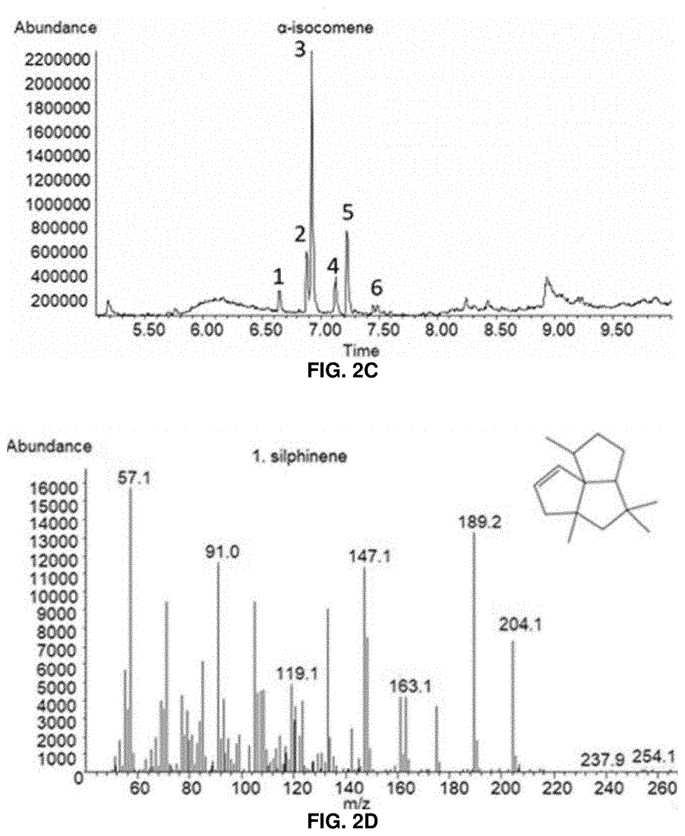

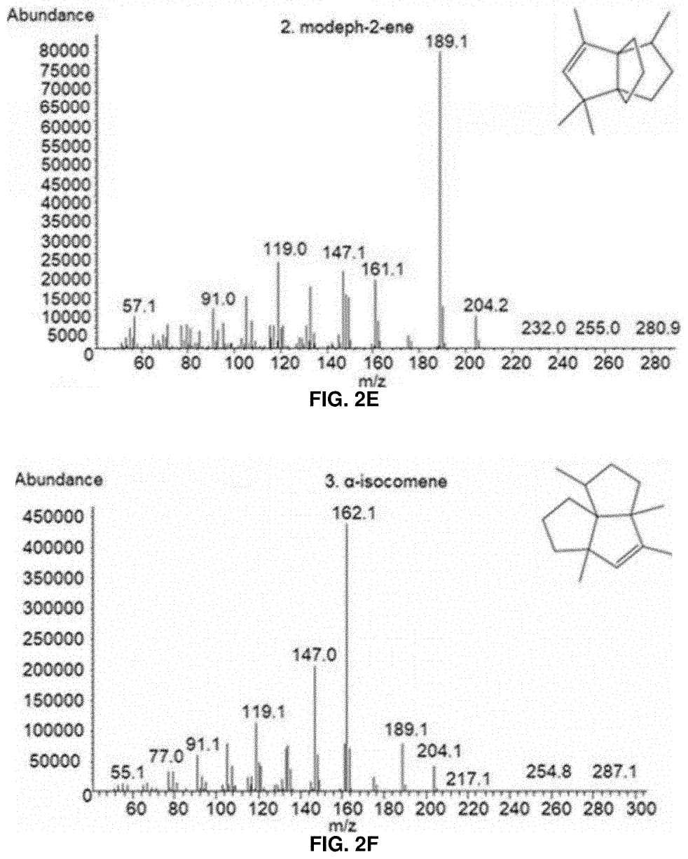

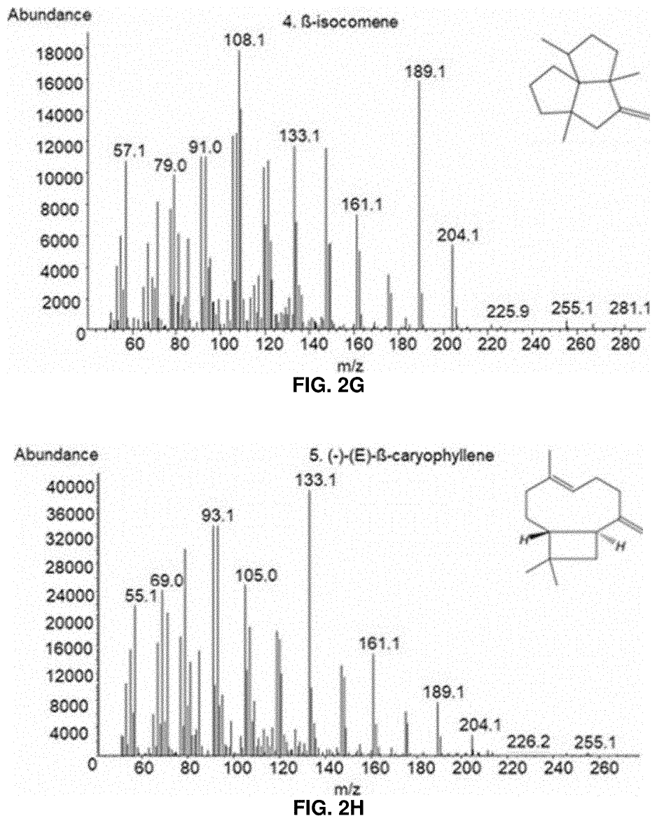

E. coli has an endogenous isoprenoid pathway (MEP pathway) that produces FPP, a substrate for sesquiterpene synthases, even though the natural level of FPP is quite low [29]. To confirm the in vivo activity of these three sesquiterpene synthases, strains harboring each plasmid are cultured expressing these enzymes under control of P_{trc}. A decane overlay is used for in situ extraction of the product, and the collected decane layer is analyzed using GC-MS. Cultures expressing coEIZS and coPentS are found to produce single products which showed fragmentation pattern of epi-isozizaene and pentalenene, respectively (FIGS. 2A and 2B). On the other hand, the culture expressing coMrTPS2 produces a broad spectrum of compounds, including α-isocomene, silphinene, modeph-2-ene, β-isocomene, (-)-(E)-β-caryophyllene and α-humulene, with α-isocomene being the major product (FIGS. 2C to 2I).

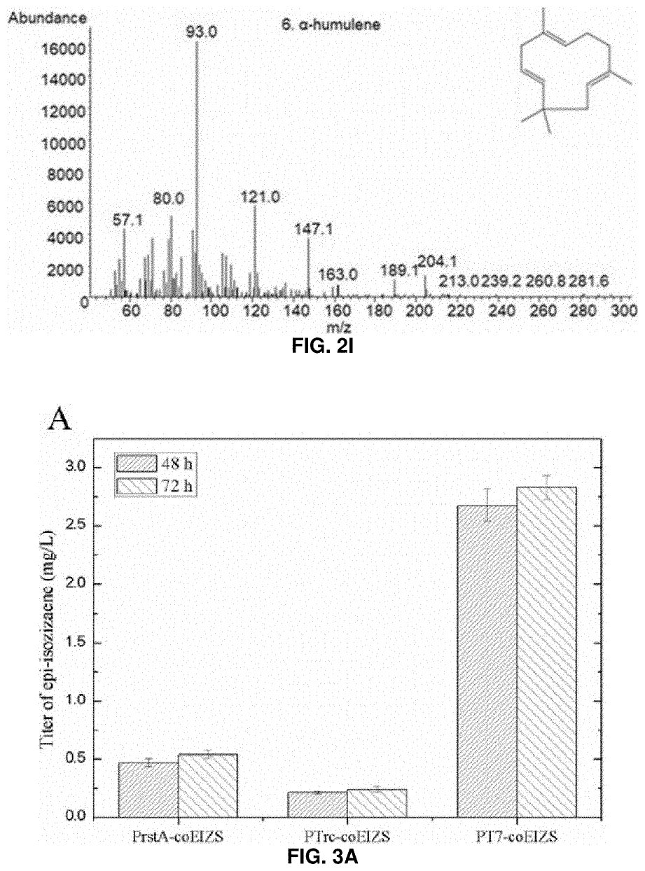

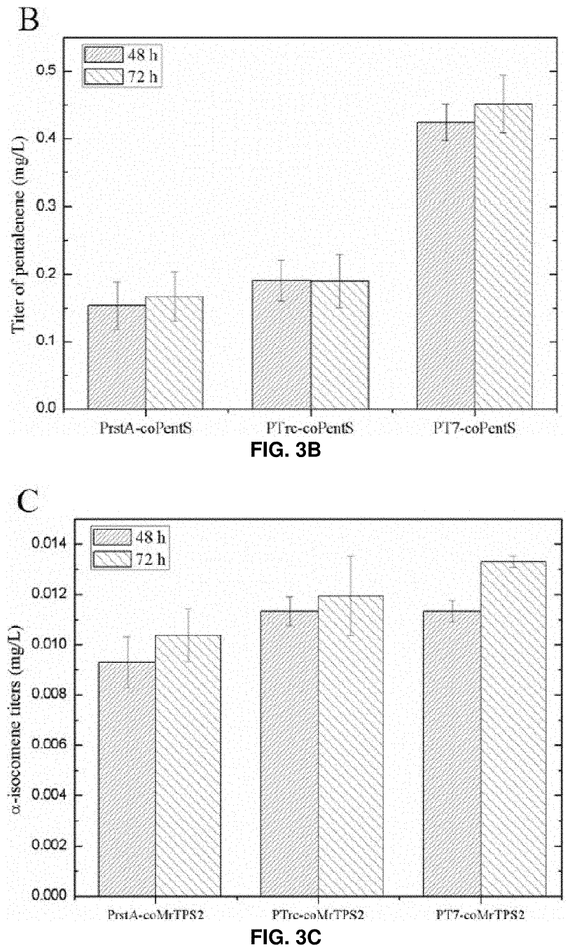

To enhance sesquiterpene production, the expression levels of the genes encoding coEIZS, coPentS and coMrTPS2 are increased using a stronger inducible promoter (FIGS. 3A to 3C). The use of the T7 promoter (P_{T7}) significantly improves biosynthesis of epi-isozizaene and pentalenene compared to strains harboring a plasmid with P_{trc}-driven terpene synthases, probably due to increased terpene synthase levels. The titers of epi-isozizaene and pentalenene improve 11.8-fold and 2.4-fold to 2.82 mg/L and 0.45 mg/L, respectively after 48 hours. Interestingly, no significant titer improvement is observed for cells harboring the P_{T7}-driven α-isocomene synthase; the titer is still more than an order of magnitude lower than those of pentalenene and epi-isozizaene, which is probably due to low enzyme activity of coMrTPS2.

### High Titer Sesquiterpene Production Using the Heterologous Mevalonate Pathway

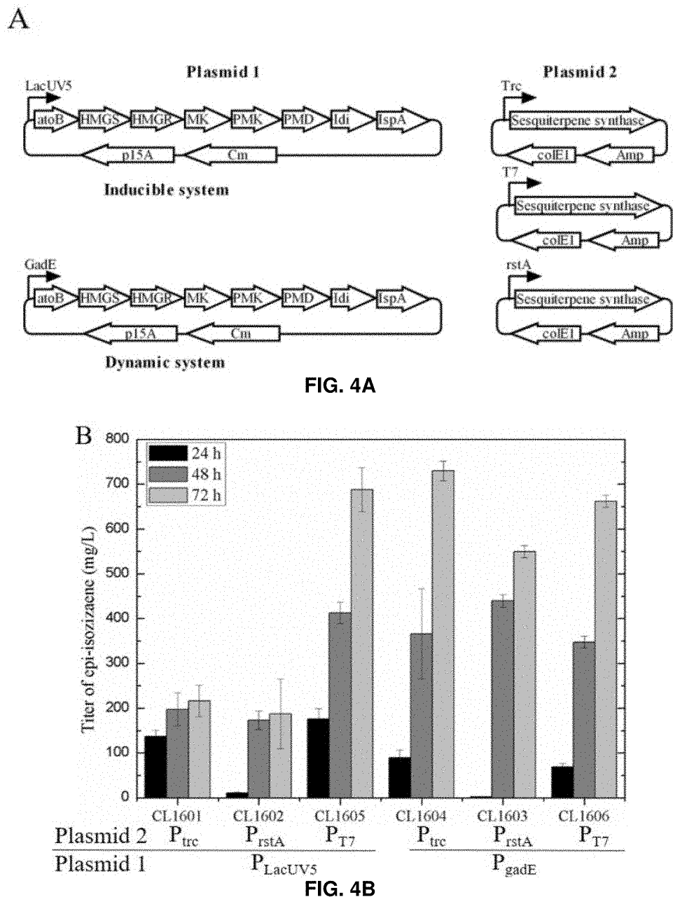

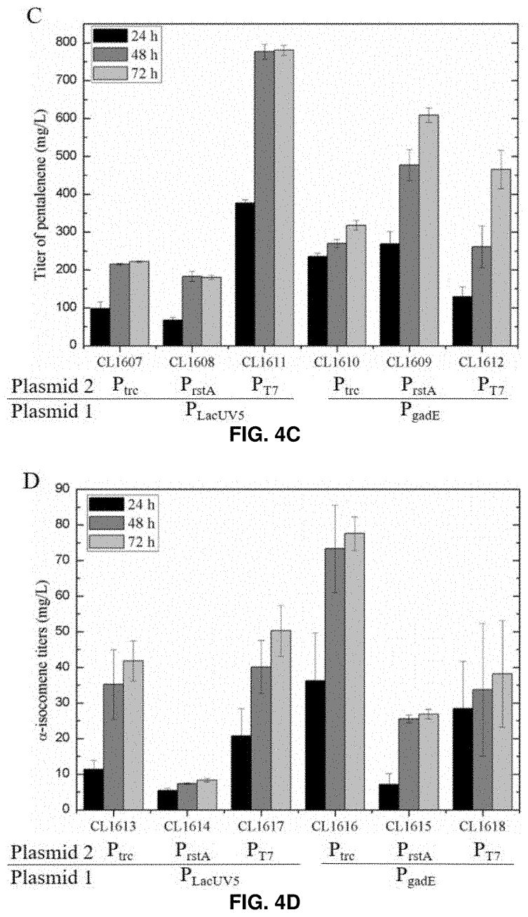

The native MEP pathway is tightly controlled by endogenous regulation and may not supply sufficient FPP for high titer sesquiterpene biosynthesis [8]. A generally low production level of sesquiterpenes with the native MEP pathway in E. coli is observed when terpene synthases are overexpressed. Previous studies showed that terpene production can be dramatically improved by introducing a heterologous mevalonate (MVA) pathway as the flux to FPP is significantly increased with the heterologous pathway [7, 30]. The eight-enzyme MVA pathway converts acetyl-CoA into FPP [30], and a system in which the genes encoding those enzymes are harbored on a single medium-copy plasmid (“plasmid 1”) and the terpene synthase is harbored on a high-copy plasmid (“plasmid 2”) is well established (FIG. 4A) [31].

An inducible promoter system is used to express MVA pathway enzymes and the terpene synthase. The “plasmid 1” containing MVA pathway genes under IPTG-inducible promoter (P_{lacUV5}) and “plasmid 2” containing the terpene synthase under control of either P_{trc} or P_{T7} are co-transformed into E. coli DH1. This results in an “inducible” sesquiterpene production systems: strain CL1601 and CL1605 for epi-isozizaene production, CL1607 and CL1611 for pentalenene production, CL1613 and CL1617 for α-isocomene production (Table 1).

In general, expression of the MVA pathway results in considerable production of target sesquiterpenes. As shown in FIGS. 4B and 4C, the titers of epi-isozizaene and pentalenene are significantly increased to 215.8 mg/L and 221.7 mg/L after 72 hours in P_{trc} strains CL1601 and CL1607 (i.e. P_{trc} is driving sesquiterpene synthases), respectively, compared to those without the MVA pathway. When P_{T7} is driving the sesquiterpene synthases in strains CL1605 and CL1612, the titers increase by 3.2-fold and 3.5-fold over those strains under P_{trc}, respectively. For α-isocomene biosynthesis, the titer increase achieved by swapping terpene synthase promoters is much smaller than values observed for epi-isozizaene. The α-isocomene titer is 41.8 mg/L in strain CL1613 when P_{Trc} is used to drive expression of MrTPS2; the titer increases to 50.2 mg/L in strain CL1617 in which P_{T7} drives MrTPS2. In general, these results confirm that a heterologously expressed MVA pathway is much more efficient in sesquiterpene production than the endogenous MEP pathway. Regarding significant titer improvements observed when terpene synthases are driven by P_{T7}, that the strong pulling effect by high terpene synthase levels driven by P_{T7} are reasoned may provide a driving force for the entire pathway by depleting the intermediate metabolite as substrates for the corresponding enzymes as well as by relieving the inhibition of mevalonate kinase by FPP. The level of coEIZS in strain CL1605 is much higher than that in strain CL1601, and the FPP level in strain CL1605 is lower than that in CL1601. This negative correlation indicates the high terpene synthase levels achieved by stronger, P_{T7}-driven coEIZS gene expression may significantly rewire pathway flux towards the conversion of FPP into epi-isozizaene.

### Introduction of the FPP-responsive Dynamic System for Sesquiterpene Production

The overproduction of FPP using an inducible heterologous MVA pathway substantially improve sesquiterpene titers. Inducible promoters, however, cannot regulate the intracellular FPP level in response to changes of cell growth or environmental conditions, and they may lead to suboptimal intracellular FPP concentrations, which can be toxic to cells [25]. In addition to the lack of dynamic control, the conventional inducible promoters require both, the use of expensive chemicals such as IPTG as inducer and human supervision for induction timing. These make the inducible systems less economically feasible in a working industrial setting. To address these challenges, two FPP-responsive promoters P_{gadE} and P_{rstA} were previously found to be useful in creating an auto-inducible isoprenoid production system [25]. The gadE promoter (P_{gadE}) down-regulates expression of genes under its control when the FPP level is high, while the rstA promoter (P_{rstA}) up-regulates gene expression at high FPP levels. Therefore, using P_{gadE} for expression of FPP-producing enzymes and P_{rstA} for expression of FPP-utilizing enzymes results in a dynamic system to control FPP levels and produce sesquiterpenes at high titers [25]. The effects of P_{gadE} and P_{rstA} on tricyclic sesquiterpene production are studied.

Plasmids expressing each terpene synthase under control of P_{rstA} are first constructed and their expression is confirmed by testing terpene production using the endogenous MEP pathway. 0.54 mg/L epi-isozizaene, 0.19 mg/L pentalenene and 0.01 mg/L α-isocomene are produced without adding any inducer; these levels are comparable to the production via the strain with terpene synthases under IPTG-inducible P_{trc} (FIGS. 3A to 3C). Then the IPTG-inducible MVA pathway is co-expressed with each of three terpene synthases under control of P_{rstA} (strains CL1602, CL1608, and CL1614 for epi-isozizaene, pentalenene, and α-isocomene production, respectively). The sesquiterpene production of these strains is compared with that from strains with terpene synthases under IPTG-inducible promoters (P_{trc} and P_{T7}) (FIGS. 4B to 4D). The specific titers of epi-isozizaene, pentalenene, and α-isocomene in strain CL1602, CL1608, and CL1614 are 125.9 mg/L, 180.4 mg/L, and 8.4 mg/L, respectively. The titers of epi-isozizaene and pentalenene are slightly lower than those of strains with terpene synthases under control of the IPTG-inducible P_{trc}. They are significantly lower than those from strains that used P_{T7} for terpene synthase expression. It is probably due to much lower terpene synthase level in the strains with P_{rstA} than those in the strain with P_{T7}. The titers of α-isocomene, however, are not straightforward to explain in the same way as α-isocomene producing strains show more complex products profile as shown in FIG. 2C probably due to the promiscuity of α-isocomene synthase. But the general trend could still be explained by much lower terpene synthase level in the strains with P_{rstA} than those in the strain with P_{T7}.

An FPP-responsive promoter P_{gadE} is introduced for the MVA pathway gene expression to build a complete dynamic system for sesquiterpene production as previously reported [25]. Six strains for each sesquiterpene synthase are prepared by combining two MVA pathway plasmids (either IPTG-inducible or FPP-responsive) and three terpene synthase plasmids (under either IPTG-inducible (P_{trc} and P_{T7}) or FPP-responsive rstA, (P_{rstA} promoters) as shown in Table 1; CL1601˜CL1606 for epi-isozizaene production, CL1607˜CL1612 for pentalenene production, and CL1613˜CL1618 for α-isocomene production.

In contrast to the IPTG-inducible MVA pathway for epi-isozizaene (CL1601, CL1602, CL1605) and pentalenene (CL1607, CL1608, CL1611) production, the use of dynamic promoter for the expression of the MVA pathway results in product titers that are less dependent on the type of promoter driving the terpene synthase (FIGS. 4B and 4C). When the IPTG-inducible MVA pathway is used, there is a significant improvement in terpene production with strains containing the P_{T7}-driven terpene synthases over strains with P_{trc} or P_{rstA} driving epi-isozizaene and pentalenene synthase expression (FIGS. 4A to 4D). This suggests the cyclization of FPP to sesquiterpene products by the terpene synthase is the primary bottleneck in the pathway, and the activity or expression level of terpene synthase is a primary determinant of the titer when the IPTG-inducible MVA pathway is used. In contrast, when the dynamic promoter is used for FPP production via the MVA pathway for epi-isozizaene (CL1604, CL1603, CL1606) and pentalenene (CL1610, CL1609, CL1612) production, there is smaller fluctuation in terpene production among strains with three different promoters driving terpene synthase expression (FIG. 4B and 4C). This result makes sense as the dynamic promoter (P_{gadE}) can regulate the MVA pathway gene expression and therefore the metabolic flux to maintain the FPP level balanced. The combination of P_{gadE}-MVA with P_{rstA}- or P_{trc}-driven sesquiterpene synthase enables the cell to balance FPP production and consumption and therefore to auto-regulate the levels of this toxic intermediate as proven by the measured intracellular FPP levels. The combination of P_{T7}-driven sesquiterpene synthases with P_{gadE}-MVA also achieves a balanced flux and considerable epi-isozizaene titer, but it is considered mainly due to the high expression level of coEIZS. Because the FPP-responsive dynamic system auto-regulates the production pathway in case of FPP depletion or FPP over-accumulation, the dynamically controlled system can improve sesquiterpene production through balanced FPP levels.

For epi-isozizaene production, three strains with dynamically regulated MVA pathway shows comparable titers to the highest producing inducible system (CL1605) after 48 hours, but their production level at 24 hours are about half of those with the inducible MVA pathway (FIG. 4B). This trend, however, does not apply to pentalenene-producing system. For pentalenene production, the highest titer is achieved using strain CL1611 which has the MVA pathway under control of P_{lacUV5} and coPentS under control of P_{T7}. The inducible system with P_{T7} driving the terpene synthase (CL1611) shows a significantly higher titer than the other pentalenene strains. With the exception of CL1611, the titers at 24 hours are more than 2-fold higher in strains with a dynamically regulated MVA pathway than in those with the inducible MVA pathway. It is an interesting result and is probably caused by the difference in the activity and the availability of two terpene synthases. Since FPP is a toxic intermediate, the FPP consumption efficiency by terpene synthase affects the balance of the entire pathway as well as the titer. The kinetics of EIZS and PentS are not available, but the differences in the activity and the relative amount of soluble enzyme shown in the SDS-PAGE results of these two terpene synthases would be responsible for the different trends in FPP accumulation and production formation in these strains.

Strain CL1609 has the full dynamic system and produces the second highest pentalenene titer and the highest OD₆₀₀. This indicates more sensitive stress-responsive dynamic regulation in CL1609. The cell growth of CL1610 and CL1612 restricts compound biosynthesis. For α-isocomene production, the worst performer is strain CL1614 containing P_{lacuv5}-MVA and P_{rstA}-coMrTPS2, probably due to the low level of terpene synthase expression under weak promoter (P_{rstA}) leading to intracellular FPP accumulation and the MVA pathway expression unbalance. This unbalance is avoided in strain CL1615 by the dynamic regulation of the MVA pathway under P_{gadE}, and this strain shows a decent level of product. Interestingly, the best producer is strain CL1616, which contains the dynamically controlled MVA pathway and the IPTG-inducible terpene synthase (P_{trc}-coMrTPS2). A similar system with a stronger IPTG-inducible promoter driving the terpene synthase (CL1618) shows a comparable titer at 24 hours, but the production does not improve much after 24 hours and shows a large standard deviation. These observations suggest FPP production in CL1618 is not well-balanced for terpene production. It is possible that the initial consumption of FPP by highly abundant terpene synthase in the early production stage triggers rapid FPP biosynthesis under dynamic promoter (P_{gadE}). This, however, may result in a rapid accumulation of FPP and disrupt the MVA pathway through unidentified process to halt FPP production. In summary, when an inducible promoter (P_{lacUV5}) drives expression of the genes encoding the MVA pathway, the combination with P_{T7}-driven terpene synthase, which is expected to have the highest terpene synthase level, achieves the highest sesquiterpene production.

Combinations of the dynamic promoter (P_{gadE})-driven MVA pathway and gene coEIZS or coPentS driven by either inducible or dynamic promoters result in reasonably high epi-isozizaene and pentalenene titers regardless of terpene synthase promoter strength, showcasing the advantages of a dynamic systems over an inducible system. Although the fully dynamic system containing both FPP-responsive promoters (P_{gadE} and P_{rstA}) do not lead to the highest production, large amounts of sesquiterpenes are still obtained for epi-isozizaene (549.7 mg/L) and pentalenene (608.5 mg/L).

### Omics-aided Evaluation of IPTG-inducible and Dynamic Promoter Systems

MVA pathway enzyme expression and metabolite production change in response to the dynamic promoters compared to the IPTG-inducible system. Epi-isozizaene production strains with 4 different combinations of promoters (strains CL1601 and CL1604 with P_{trc}-coEIZS, and CL1605 and CL1606 with P_{T7}-coEIZS; Table 1) have their metabolomics and proteomics data collected at six time points over 48 hours. The concentrations of acetyl-CoA decreases after 4 hours in all four strains, and MVA concentrations, which are the highest among the pathway metabolites monitored, increases at early production phases. MVA levels then slowly decreases in all four strains, but MVA concentration reaches much higher level in strains with P_{T7} (CL1605 and CL1606) than the other strains before it starts to decrease. Interestingly, while the MVA levels in strain CL1605 are almost 20 times higher than that in strain CL1601 at the late fermentation phase, other metabolites such as Mev-P, IPP/DMAPP, and FPP show similar levels in CL1601 and CL1605. Proteomics analysis shows CL1601 expresses the lowest levels of MVA pathway proteins overall among these four strains, which may explain the poor production titer of this strain. Compared to CL1601, the enzyme levels are significantly higher in CL1605. Especially for proteins AtoB, HMGS and HMGR, strain CL1605 expresses about 5-10 times more enzymes than CL1601. This may explain the markedly higher levels of MVA in CL1605. It is interesting to observe that the use of strong promoter (P_{T7}) for terpene synthase gene expression improves both the level of coEIZS enzyme as intended and the expression of upstream pathway enzymes. It is speculated this is related to the deficiency of FPP in early stage due to high level of terpene synthase. FPP is an essential metabolite for E. coli growth, and when the strain experiences a deficit of FPP, it pushes the pathway to produce more FPP for survival. As a result, the first three enzyme of the MVA pathway are expressed more. However, the next two enzymes (MK and PMK) levels remain quite low as previously observed [44] and this may limit the conversion of MVA to the next intermediates.

Dynamic changes in intracellular FPP concentrations are observed in strain CL1604 harboring P_{gadE}-MevT⁻MBIS (JBEI-2872) and P_{trc}-CoEIZS plasmids (JBEI-15862). In this case the FPP level decreases in the first 2 hours, increases between 2-6 hours, decreases again between 6-8 hours, then eventually keeps slowly accumulating in the cell. This observation is consistent with previous findings [25]. However, strain CL1606, which harbors JBEI-2872 and JBEI-15866 expresses epi-isozizaene synthase under a strong promoter (P_{T7}), where FPP concentration is constantly maintained at very low level. This behavior may result from the strong promoter (P_{T7}) expressing excessive epi-isozizaene synthase, which depletes intracellular FPP in CL1606. The low FPP level de-represses P_{gadE} and makes it behave constitutively in CL1606. This is consistent with generally higher levels of the first three enzymes, which are directly influenced by the promoter strength, in strain CL1606 over strain CL1604.

Large Scale Production, Purification and NMR Characterization of the Biosynthetic Sesquiterpenes

Epi-isozizaene and pentalenene are not commercially available in large quantities. Experimental testing of these compounds for jet fuel specification, however, requires a large quantity of these compounds. Therefore, it is required to demonstrate a larger-scale fermentation of these sesquiterpenes producers to achieve this goal. To demonstrate scalability and the downstream purification process, a large-scale production of these jet fuel precursors in 4-L flasks with batch culture (TB medium with 0.4% glycerol) is performed using the inducer-free dynamic E. coli strains CL1603 and CL1609 for epi-isozizaene and pentalenene, respectively. During large-scale production, the cultures are overlayed with 20% nonane instead of decane to facilitate sesquiterpene extraction, as the boiling temperature of nonane (151° C.) is lower than that of decane or dodecane (174.1° C. or 216.2° C., respectively), which makes it easier to recover the product from the overlay by evaporation of the solvent.

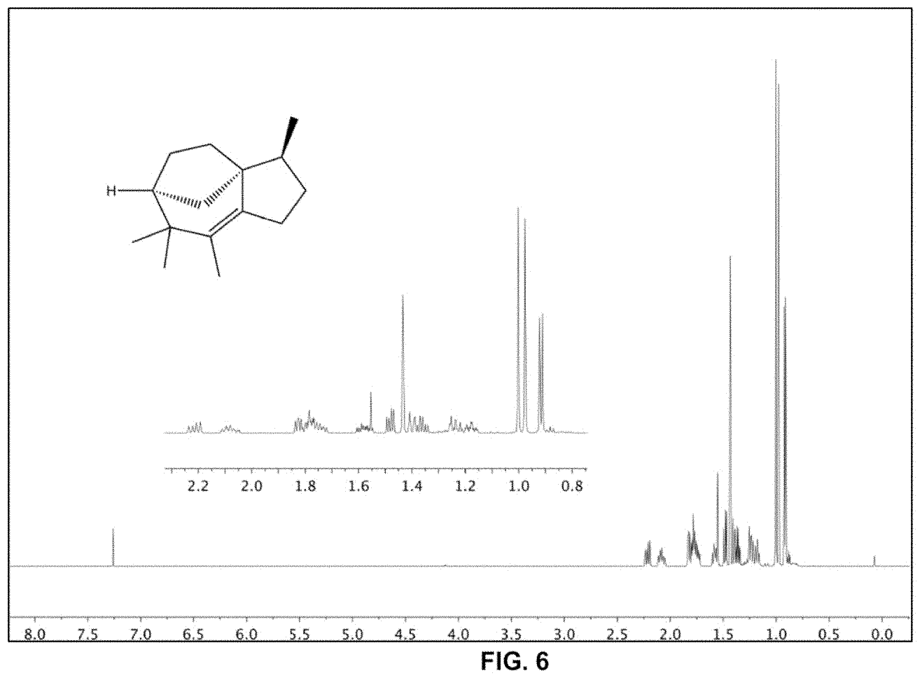

About 0.8 L and 0.5 L of nonane are collected from the epi-isozizaene biosynthesis cultures and the pentalenene biosynthesis cultures, respectively, using a separatory funnel. The products are recovered from the nonane overlay by a series of purification steps as described in the METHODS section. NMR is used to identify the product and check its purity (i.e. the specificity of terpene synthase toward the expected product) which confirms these two compounds with the purities over 90% and assigned their NMR spectra (FIGS. 6 to 9).

Finally, about 1 mL epi-isozizaene and 0.66 mL pentalenene with over 90% purity is prepared from large-scale batch cultures (4 L for epi-isozizaene and 2.5 L for pentalenene), which contains 1.08 g epi-isozizaene and 0.66 g pentalenene. The recovery efficiencies are about 49% and 43%, respectively, when considering the production titer from GC analysis.

### Sesquiterpene Production in Yeast

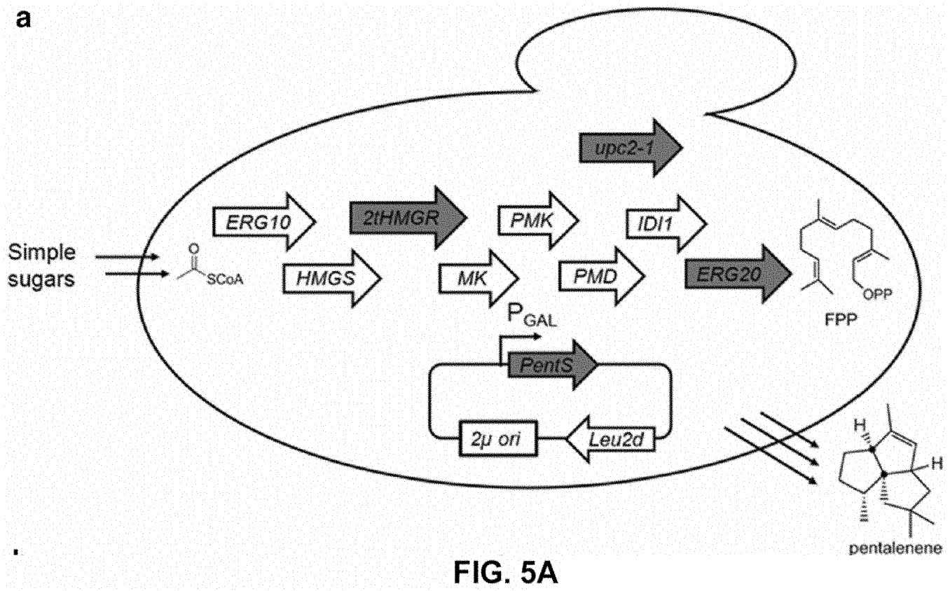

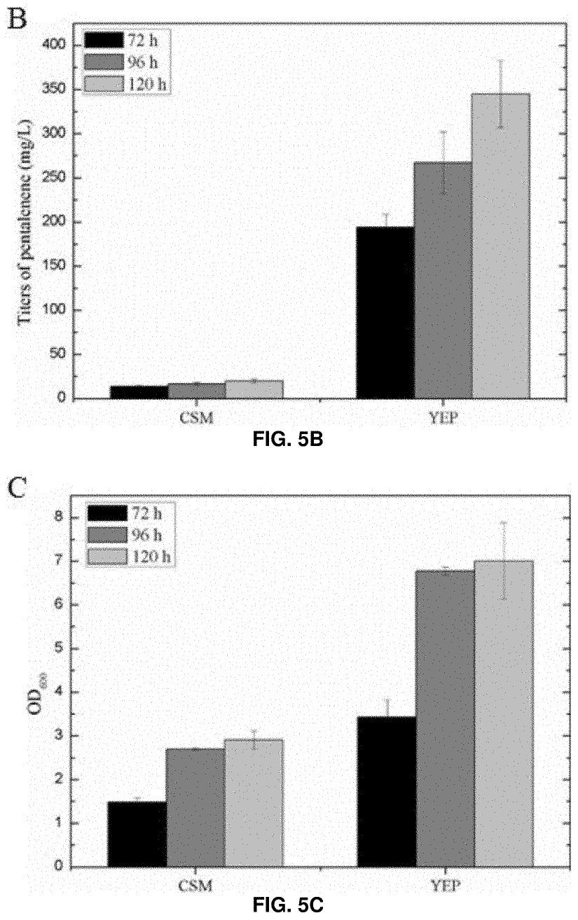

The Baker's yeast, Saccharomyces cerevisiae has been the host of choice for industrial production of various bio-products and widely used for terpene biosynthesis. Therefore, in addition to production in E. coli, it is sought to demonstrate the production of one of these sesquiterpenes, pentalenene in S. cerevisiae (FIGS. 5A to 5C).

A previously reported host strain is engineered for high FPP production (EPY300) [32] (FIG. 5A). To efficiently convert FPP to pentalenene, the codon-optimized gene coPentS under the control of a galactose promoter is introduced on a high-copy plasmid (2 μ) containing the auxotrophic Leu2d marker, as previously used to achieve high levels of amorphadiene [33]. Defined CSM medium and non-selective, rich, mixed carbon YEP medium supplemented with 1.8% galactose/0.2% glucose are used for culturing the yeast cells. Consequently, higher cell density and production are achieved using the rich medium. After 120 hours, 344.7 mg/L pentalenene is obtained in mixed carbon YEP medium, which is 17.5-fold higher than that in CSM medium (FIG. 5B).

### Discussion

Three tricyclic sesquiterpenes are promising jet fuel precursors. Microbes are engineered to produce these jet fuel precursors at high titers to demonstrate the potential of microbial jet fuel manufacturing. In particular, an inducer-free system using previously identified FPP-responsive promoters is developed and produces a respectable amount of sesquiterpenes, allowing application to the industrial fermentation process.

The engineered MVA pathway is a very efficient FPP supply platform for sesquiterpene biosynthesis, and the increased FPP flux drives the implementation of FPP-responsive promoters (P_{gadE} and P_{rstA}) to render higher production titers by relieving the toxicity from FPP accumulation, as previously reported [25]. This dynamic setting, however, does not always result in higher titer compared to the inducible system (with P_{lacUV5} and P_{T7} promoters) used as described herein, and this implies that there are more factors that affect efficiency of the dynamically regulated system and it may require additional adjustment to achieve balanced enzyme expression and optimized pathway performance. Finally, the production of one of these sesquiterpenes in S. cerevisiae, a microbial host that is widely used for biofuel production, is demonstrated. Much work including terpene synthase enzyme engineering (for a better activity and specificity) and culture medium and process optimization (for scale-up) would be needed to improve the titer, yield, and productivity of these sesquiterpenes in S. cerevisiae.

Tricyclic sesquiterpenes are expected to have several advantages over the already commercialized acyclic bio-jet fuel precursor, farnesene (or Biofene® from Amyris, Inc., Emeryville, Calif.) as versatile feedstocks to jet fuels. First, they have higher densities than farnesene (around 1.0 g/mL vs 0.81 g/mL of farnesene), which deliver higher volumetric net heats of combustion [13, 34]. Considering small increases in volumetric energy density can have major impacts on profitability of airlines, especially for long haul flights, higher density fuel would be preferred over lower density fuels. Also, while farnesene has four double bonds and requires four molar equivalents of dihydrogen to generate the actual fuel molecule (saturated hydrocarbons, farnesanes), tricyclic sesquiterpenes have only one double bond and require just one dihydrogen molecule per fuel molecule, making the refining process less expensive.

In the past decade, the farnesene biosynthetic pathway has been extensively studied and optimized [35-37]. Currently a production of $2.2/kg has been achieved and the estimated price will become even lower in the near future [38]. Since tricyclic sesquiterpenes and farnesene share a large portion of their biosynthetic pathways, it is expect that an engineering strategy similar to that used to produce farnesene could be applied to achieve high productivity and yield of tricyclic terpenes.

### Conclusions

Sesquiterpenes and sesquiterpene-derived molecules are promising alternatives to aviation fuels. It is proposed tricyclic sesquiterpene compounds, epi-isozizaene, pentalenene and α-isocomene as jet fuel precursors to expand the scope of bio-jet fuels. The sesquiterpene synthases are introduced into E. coli with either endogenously or heterologously expressed isoprenoid pathways, and the production pathway is engineered to increase the product titer. IPTG-inducible and dynamically-controlled systems are compared for sesquiterpene production; proteomics and metabolomics analysis of these systems explain the different behaviors of various strains. In addition, a yeast strain is generated for one of the jet fuel targets, pentalenene, to explore an alternate host for large scale fermentation. In summary, the engineered strains produce tricyclic sesquiterpenes at high titers with 727.9 mg/L epi-isozizaene, 780.3 mg/L pentalenene and 77.5 mg/L α-isocomene in E. coli in a batch culture with 10 g/L glucose, and 344 mg/L pentalenene in S. cerevisiae with YPG medium. This provides a solid ground toward sustainable alternatives to high energy density fuels for the long-term global energy demand.

### Methods

### Plasmids and Strains Construction

All plasmids and strains described herein are available on the JBEI public registry (the website for public-registry.jbei.org) and listed in Table 1 along with a brief description of production strains. E. coli DH10B is used for plasmid construction, and E. coli DH1 and E. coli DH1 (DE3) are used as hosts for terpene production and protein expression. Plasmids containing eight genes for the mevalonate pathway (atoB, hmgs, hmgr, mk, pmk, pmd, idi, ispA) are introduced into E. coli host, enabling production of FPP from acetyl-CoA. Five genes (hmgs, hmgr, mk, pmk, pmd) originate from S. cerevisiae, and three genes (atoB, idi, ispA) are native E. coli genes. For sesquiterpene production in E. coli, four terpene synthase genes (the original EIZS from Streptomyces and three E. coli codon-optimized genes, coEIZS, coPentS and coMrTPS2, which are amplified from gBlock templates synthesized by IDT (Iowa, USA)) are cloned into pTrc99 at NcoI and XbaI sites, to yield the plasmids JBEI-15857, JBEI-15862, JBEI-15867 and JBEI-15865, respectively. To insert an N-terminal His-tag for protein purification, PCR is performed using the primers that included a His-tag sequence and the PCR product is cloned into pTrc99 vector yielding plasmids JBEI-15856, JBEI-15855, JBEI-15854 and JBEI-15853, respectively. Genes coEIZS, coPentS and coMrTPS2 are cloned into vector pPrstA-RFP [25] via the isothermal assembly method [39] using a commercial Gibson Assembly® Master Mix kit (New England Biolabs, Ipswich, Mass.) to yield plasmids prstA-coEIZS, prstA-coPentS and prstA-coMrTPS2, respectively. Plasmid pBbE7a-coEIZS, pBbE7a-coPentS and pBbE7a-coMrTPS2 are also constructed using vector pBbA7a-RFP between NdeI and XhoI sites. For pentalenene production in S. cerevisiae, coPentS is cloned into vector pRSLeu2d [31] between the NheI and XhoI sites.

TABLE 1
Description of E. coli base strains,
plasmids and gene clusters described herein.

| Plasmid | | | |
|---|---|---|---|
| reference | Plasmid name | Relevant genotype | Reference |
| JBEI-2704 | placUV5- | p15A, CmR | [44] |
| | MevT-MBIS | | |
| JBEI-2872 | pgadE- | p15A, CmR | [25] |
| | MevT-MBIS | | |
| JBEI-15857 | pTrc99a-EIZS | ColE1(pBR322) ori, | |
| | | AmpR | |
| JBEI-15862 | pTrc99a- | ColE1(pBR322) ori, | |
| | coEIZS | AmpR | |
| JBEI-15867 | pTrc99a- | ColE1(pBR322) ori, | |
| | coPentS | AmpR | |
| JBEI-15865 | pTrc99a- | ColE1(pBR322) ori, | |
| | coMrTPS2 | AmpR | |
| JBEI-15856 | pTrc99a-his- | ColE1(pBR322) ori, | |
| | EIZS | AmpR, his tag | |
| JBEI-15855 | pTrc99a-his- | ColE1(pBR322) ori, | |
| | coEIZS | AmpR, his tag | |
| JBEI-15854 | pTrc99a-his- | ColE1(pBR322) ori, | |
| | coPentS | AmpR, his tag | |
| JBEI-15853 | pTrc99a-his- | ColE1(pBR322) ori, | |
| | coMrTPS2 | AmpR, his tag | |
| JBEI-15849 | prstA-coEIZS | ColE1(pBR322) ori, | |
| | | AmpR | |
| JBEI-15863 | prstA- | ColE1(pBR322) ori, | |
| | coPentS | AmpR | |
| JBEI-15864 | prstA- | ColE1(pBR322) ori, | |
| | coMrTPS2 | AmpR | |
| JBEI-15866 | pBbE7a- | ColE1(pBR322) ori, | |
| | coEIZS | AmpR | |
| JBEI-15858 | pBbE7a- | ColE1(pBR322) ori, | |
| | coPentS | AmpR | |
| JBEI-15859 | pBbE7a- | ColE1(pBR322) ori, | |
| | coMrTPS2 | AmpR | |
| JBEI-15861 | pRSLeu2d- | 2μ ori, pBR322 ori, | |
| | coPentS | Leu2d | |

| Strain name | Description | Reference |
|---|---|---|
| CL1601 | JBEI-2704 + JBEI-15862 | |
| CL1602 | JBEI-2704 + JBEI-15849 | |
| CL1603 | JBEI-2872 + JBEI-15849 | |
| CL1604 | JBEI-2872 + JBEI-15862 | |
| CL1605 | JBEI-2704 + JBEI-15866 | |
| CL1606 | JBEI-2872 + JBEI-15866 | |
| CL1607 | JBEI-2704 + JBEI-15867 | |
| CL1608 | JBEI-2704 + JBEI-15863 | |
| CL1609 | JBEI-2872 + JBEI-15863 | |
| CL1610 | JBEI-2872 + JBEI-15867 | |
| CL1611 | JBEI-2704 + JBEI-15858 | |
| CL1612 | JBEI-2872 + JBEI-15858 | |
| CL1613 | JBEI-2704 + JBEI-15865 | |
| CL1614 | JBEI-2704 + JBEI-15864 | |
| CL1615 | JBEI-2872 + JBEI-15864 | |
| CL1616 | JBEI-2872 + JBEI-15865 | |
| CL1617 | JBEI-2704 + JBEI-15859 | |
| CL1618 | JBEI-2872 + JBEI-15859 | |
| CL1619 | S. cerevisiae EPY300 | |
| | harboring JBEI-15861 | |

Sesquiterpene Production in E. coli

For MVA pathway expression, E. coli DH1 is co-transformed with an MVA pathway plasmid JBEI-2704 (for the IPTG-inducible system) or JBEI-2872 (for the dynamic system) and a plasmid harboring one of each terpene synthase gene. Pre-cultures of E. coli strains are diluted 1:100 into EZ rich defined medium (Teknova, Hollister, Calif.) containing 10 g/L glucose as carbon source and ampicillin (100 μg/mL) and chloramphenicol (30 μg/mL) for plasmid maintenance. The cell cultures are grown at 37° C. until OD₆₀₀ reached ˜0.6, and induced with 0.5 mM IPTG while shaking at 30° C.; 20% decane is added for in situ product extraction. At 24 h, 48 h and 72 h time points, 10 μL of the decane layer are taken and diluted into 990 μL of ethyl acetate for GC-MS analysis. Caryophyllene is used as an internal standard for the production of epi-isozizaene and pentalenene, while pentalenene is used as internal standard for the production of α-isocomene since the strain with MrTPS2 also produce detectable amount of caryophyllene.

Detection of Sesquiterpenes with GC-MS

The decane fraction from the culture medium is analyzed by GC-MS (Thermo Trace Ultra with PolarisQ MS) with a TR-5MS column (30 m×0.25 mm ID×0.25 μm film) using the following conditions: inlet at 250° C., 1.1 mL min⁻¹ constant flow, transfer line at 300° C., ion source at 200° C., scan m/z 50-300. Oven: 100° C. for 4 min, ramp at 30° C. min⁻¹ to 250° C., hold for 1 min. Samples of 1 μL are injected into the GC. The products are analyzed in total ion monitoring mode and selective ion monitoring mode (m/z 204).

### Large Scale Production, Purification, and Characterization of the Biosynthetic Sesquiterpenes

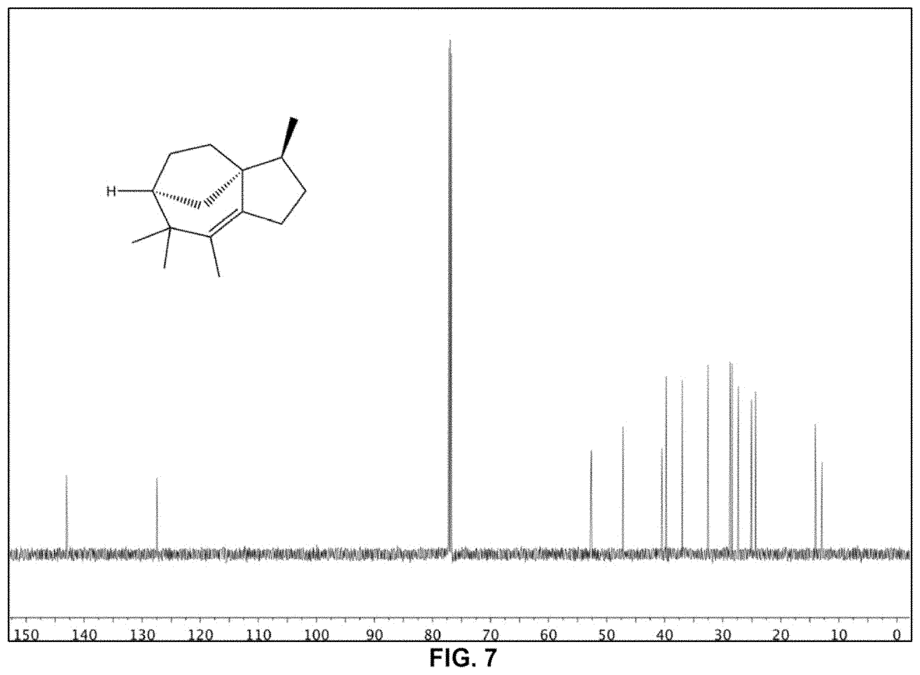

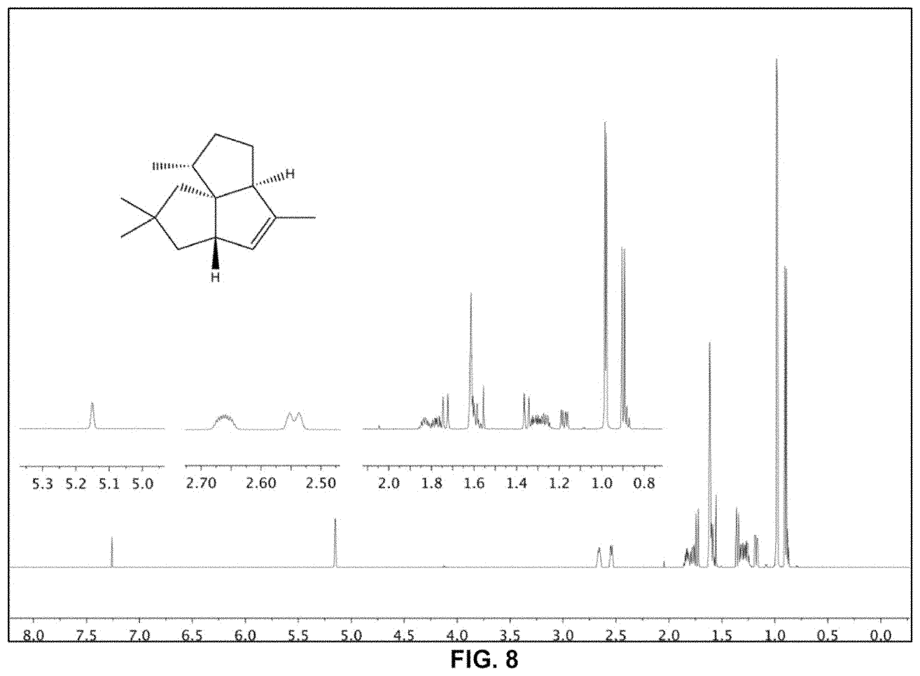

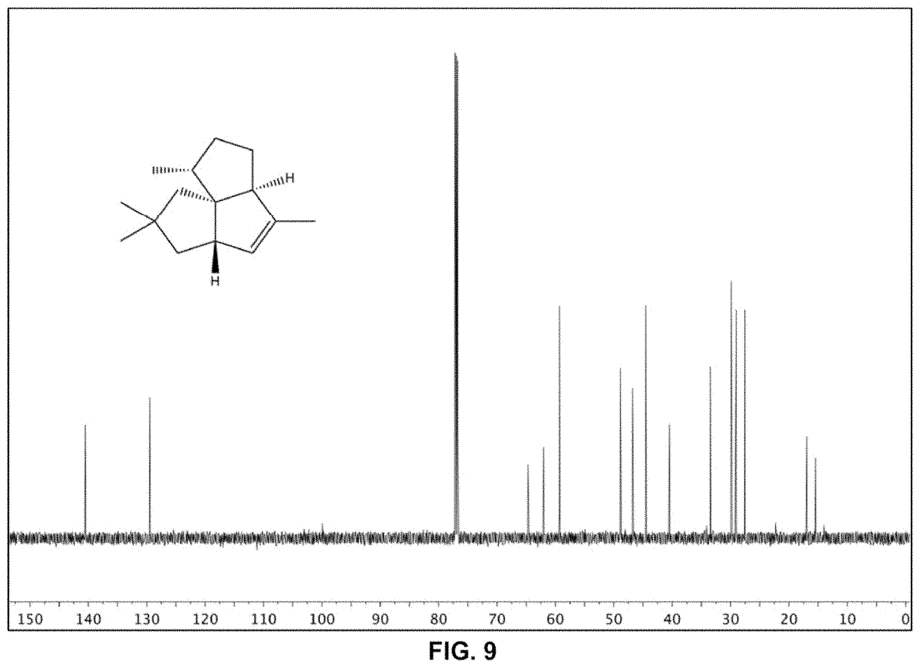

For the preparation of sesquiterpene compounds epi-isozizaene and pentalenene, the E. coli strains for sesquiterpene production are cultured at 30° C. for 72 hours after induction. One-liter of Terrific Broth (Difco) with 4% glycerol is used for batch production in a 4-liter flask overlayed with 200 mL of nonane to facilitate sesquiterpene extraction. The nonane overlay is collected from the culture medium using a separatory funnel and dried over sodium sulfate. Nonane is evaporated under reduced pressure, and the crude sesquiterpenes are loaded onto a silica gel column for column chromatography using hexanes as the mobile phase. The product containing fractions are pooled and the solvent is evaporated under reduced pressure. The product is obtained as colorless oil and further purified by preparative silver ion thin-layer chromatography (Ag-TLC) for nuclear magnetic resonance (NMR) analysis. Silver nitrate is adsorbed onto thin-layer silica by methods described previously [40]. TLC plates (500 μm layer thickness) are sprayed with an AgNO₃ solution (5% w/v in methanol), dried at 110° C. for 10 minutes and allowed to cool at room temperature directly before use. Plates are developed in hexane and the pure product is dissolved in n-pentane and concentrated under reduced pressure. The structure of the isolated epi-isozizaene and pentalenene dissolved in CDCl₃ are analyzed by 1H and 13C NMR using a 500 MHz NMR spectrometer (Bruker DRX-500, Bruker Corp.. Billerica, Mass.). NMR spectrum of biosynthetic epi-isozizaene: ¹H NMR (600 MHz, CDCl₃): δ2.22 (ddp, J=17.0, 9.1, 1.2 Hz, 1H), 2.13-2.03 (m, 1H), 1.83 (dd, J=7.4, 5.3 Hz, 1H), 1.80-1.76 (m, 2H), 1.76-1.71 (m, 1H), 1.62-1.54 (m, 1H), 1.48 (dd, J=10.5, 5.3 Hz, 1H), 1.43 (t, J=1.5 Hz, 3H), 1.40 (d, J=10.0, 1.9 Hz, 1H), 1.38-1.34 (m, 1H), 1.26-1.21 (m, 1H), 1.18 (tdd, J=11.5, 3.4, 2.1 Hz, 1H), 1.00 (s, 3H), 0.98 (s, 3H), 0.92 (d, J=6.4 Hz, 3H) (FIG. 6). ¹³C NMR (151 MHz, CDCl₃): δ143.0, 127.5, 52.7, 47.2, 40.5, 39.7, 37.0, 32.5, 28.7, 28.4, 27.3, 25.1, 24.4, 14.1, 12.9 (FIG. 7). NMR spectrum of biosynthetic pentalenene: ¹H NMR (600 MHz, CDCl₃): δ5.15 (h, J=1.6 Hz, 1H), 2.66 (ddh, J=9.3, 4.5, 2.1 Hz, 1H), 2.54 (d, J=9.4 Hz, 1H), 1.86-1.80 (m, 1H), 1.78 (ddd, J=12.5, 6.1, 3.0 Hz, 1H), 1.73 (dd, J=13.1, 1.0 Hz, 1H), 1.62 (h, J =1.4 Hz, 4H), 1.37-1.23 (m, 4H), 1.18 (ddd, J=12.5, 5.1, 0.9 Hz, 1H), 0.98 (s, 3H), 0.98 (s, 3H), 0.90 (d, J=7.1 Hz, 3H) (FIG. 8). ¹³C NMR (151 MHz, CDCl₃): δ140.5, 129.5, 64.7, 62.0, 59.3, 48.9, 46.8, 44.5, 40.5, 33.5, 29.9, 29.1, 27.5, 17.0, 15.5 (FIG. 9).

### Metabolite Analysis and Targeted Proteomics

All metabolites are analyzed by liquid chromatography mass spectrometry (LC-MS; Agilent Technologies 1200 Series Rapid Resolution HPLC system and Agilent Technologies 6210 time-of-flight mass spectrometer (TOF-MS) (Agilent Technologies, Santa Clara, Calif.)) on a SeQuant® ZIC®-pHILIC column (150 mm length, 2.1-mm internal diameter, and 5-μm particle size). Targeted proteomics is performed using an Agilent 1290 liquid chromatography system coupled to an Agilent 6460 QQQ mass spectrometer (Agilent Technologies, Santa Clara, Calif.) via selected reaction monitoring (SRM) at the same points of the samples of metabolites.

### Pentalenene Production in S. Cerevisiae

Pre-cultures of S. cerevisiae strain harboring the plasmid pRsLeu2d-coPentS is used to inoculate at an OD₆₀₀ of 0.05 into 5 mL of Complete Supplemental Mixture (CSM; MB Biomedicals, Solon, Ohio) medium or rich mixed carbon Yeast Extract Peptone (YEP; 1% Yeast Extract, 2% Peptone) medium supplemented with 1.8% galactose/0.2% glucose and overlayed with 10% decane. At 72 h, 96 h and 120 h, 10 μl decane layer is sampled and diluted 100 times into ethyl acetate with caryophyllene as the internal standard. For pentalenene quantification, the samples are analyzed by GC-MS as described above.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A genetically modified host cell capable of producing one or more tricyclic sesquiterpenes, said genetically modified host cell comprising one or more tricyclic sesquiterpenes synthase.

2. The genetically modified host cell of claim 1, wherein (a) the tricyclic sesquiterpene is epi-isozizaene, and the tricyclic sesquiterpene synthase is epi-isozizaene synthase (EIZS);
(b) the tricyclic sesquiterpene is pentalenene, and the tricyclic sesquiterpene synthase is pentalenene synthase (PentS); or (c) the tricyclic sesquiterpene is α-isocomene, and the tricyclic sesquiterpene synthase is α-isocomene synthase (MrTPS2); wherein the EIZS comprises (i) an amino acid sequence having at least 70% identity with SEQ ID NO:1, and (ii) the amino acid sequence of SEQ ID NO:4 or SEQ ID NO:5; the PentS comprises (i) an amino acid sequence having at least 70% identity with SEQ ID NO:2, and (ii) the amino acid sequence of SEQ ID NO:4 or SEQ ID NO:5; and the MrTPS2 comprises (i) an amino acid sequence having at least 70% identity with SEQ ID NO:3, and (ii) the amino acid sequence of SEQ ID NO:4 or SEQ ID NO:5.

3. The genetically modified host cell of claim 2, wherein the tricyclic sesquiterpene is epi-isozizaene, and the tricyclic sesquiterpene synthase is epi-isozizaene synthase (EIZS).

4. The genetically modified host cell of claim 2, wherein the tricyclic sesquiterpene is pentalenene, and the tricyclic sesquiterpene synthase is pentalenene synthase (PentS).

5. The genetically modified host cell of claim 2, wherein the tricyclic sesquiterpene is α-isocomene, and the tricyclic sesquiterpene synthase is α-isocomene synthase (MrTPS2).

6. The genetically modified host cell of claim 1, further comprising one or enzymes of the mevalonatae (MVA) pathway, wherein the MVA pathway is heterologous to the genetically modified host cell.

7. The genetically modified host cell of claim 6, further comprising acetoacetyl-CoA thiolase (AtoB), HMG-CoA synthase (HMGS), HMG-CoA reductase (HMGR), mevalonate kinase (MK), phosphomevalonate kinase (PMK), mevalonate diphosphate decarboxylase (PMD), isopentenyl diphosphate (IPP) isomerase (Idi), and farnesyl diphosphate (FPP) synthase (IspA), which are heterologous to the genetically modified host cell.

8. The genetically modified host cell of claim 7, wherein the genetically modified host cell is a bacteriium that does not naturally have the MVA pathway.

9. The genetically modified host cell of claim 8, wherein the bacterium that does not naturally have the MVA pathway is of the genus Escherichia, Enterobacter, Azotobacter, Erwinia, Bacillus, Pseudomonas, Klebsielia, Proteus, Salmonella, Serratia, Shigella, Rhizobia, Vitreoscilla, or Paracoccus.

10. The genetically modified host cell of claim 9, wherein the bacteria that does not naturally have the MVA pathway is of the genus Escherichia.

11. The genetically modified host cell of claim 10, wherein the genetically modified host cell is Escherichia coli.

12. The genetically modified host cell of claim 1, wherein the genetically modified host cell comprises an endogenous mevalonatae (MVA) pathway.

13. The genetically modified host cell of claim 12, wherein the genetically modified host cell is a yeast.

14. The genetically modified host cell of claim 13, wherein the genetically modified host cell is a yeast of the genus Saccharomyces.

15. The genetically modified host cell of claim 14, wherein the genetically modified host cell is Saccharomyces cerevisiae.

16. A method for producing one or more tricyclic sesquiterpenes comprising: (a) providing a, or a culture thereof, genetically modified host cell of claim 1, (b) culturing the genetically modified host cell to produce one or more tricyclic sesquiterpenes, (c) optionally extracting or separating the produce one or more tricyclic sesquiterpenes from the culture, (d) optionally hydrogenating the one or more tricyclic sesquiterpenes extracted or separated from the culture, and (e) optionally introducing a fuel additive to the extracted or separated one or more tricyclic sesquiterpenes.

17. A fuel composition comprising: (a) a tricyclic sesquiterpene or a hydrogenation product of a tricyclic sesquiterpene; and (b) a fuel additive.

18. The fuel composition of claim 17, wherein the fuel composition comprises a tricyclic sesquiterpene, wherein the tricyclic sesquiterpene is an epi-isozizaene, pentalenene, or α-isocomene.

19. The fuel composition of claim 17, wherein the fuel composition comprises the hydrogenation product of a tricyclic sesquiterpene, wherein the hydrogenation product of a tricyclic sesquiterpene is epi-isozizaane, pentalenane, or α-isocomane.
